(19) United States
(12) Patent Application Publication
(10) Pub. No.: US 2021/0000870 A1
(43) Pub. Date: Jan. 7, 2021

(54) BISPECIFIC CHIMERIC ANTIGEN RECEPTORS AND THEIR APPLICATION IN THE TREATMENT OF TUMOR

(71) Applicant: Cellyan Therapeutics (Wuhan) Co., LTD., Wuhan (CN)

(72) Inventors: Dan JIN, Wuhan (CN); Lu LI, Wuhan (CN); Quangao HUA, Wuhan (CN)

(21) Appl. No.: 16/705,301

(22) Filed: Dec. 6, 2019

(30) Foreign Application Priority Data
Oct. 8, 2018 (CN) 201811167700.4

(51) Int. Cl. A61K 35/17; C07K 14/725; A61K 47/65; C07K 16/28; C07K 14/705; C07K 14/73

(52) U.S. Cl. CPC A61K 35/17; C07K 14/7051; A61K 47/65; A61K 45/06; C07K 14/70578; C07K 14/70514; C07K 16/2815; C07K 16/2896

(57) ABSTRACT

The embodiments of the present invention provide a bispecific chimeric antigen receptor, consisting of a signal peptide, two specific antigen-binding fragments, an extracellular spacer region, a transmembrane region, an intracellular co-stimulatory signaling domain, the first antigen that is recognized and bound by the specific antigen-binding fragments is a member selected from the group consisting of CD19, CD20, CD22, CD33, CD269, CD138, CD79a, CD79b, CD23, ROR1, CD30, B cell surface antibody light chain, CD44, CD123, Lewis Y, CD7 and CD46; the second antigen that is recognized and bound by the specific antigen-binding fragments is CD38, the two specific antigen-binding fragments is linked by a linker peptide, the bispecific chimeric antigen receptor can recognize respectively two kinds of tumor-associated antigens by constructing low affinity chimeric antigen receptors and high affinity chimeric antigen receptors and have very strong specificity. In addition, the embodiments of the present invention also provide a use of the bispecific chimeric antigen receptor in the treatment of tumors.

### TECHNICAL FIELD

The embodiments of the present invention relate to the field of cell immunotherapy, and in particular relates to bispecific chimeric antigen receptors and their application in the treatment of tumor.

### BACKGROUND OF THE INVENTION

With the development of tumor immunology theory and clinical technology, the chimeric antigen receptor T-cell immunotherapy (CAR-T) has become one of the most promising tumor immunotherapies. The chimeric antigen receptor CAR consists of a tumor-associated antigen binding region, an extracellular hinge region, a transmembrane region, and an intracellular signal transduction region. The CAR-T cell therapy expresses the single chain fragment variable (scFv) that recognizes tumor-associated antigen and the fusion protein of T cell activation sequence onto the surface of T cells by exogenous gene transfection technology, so that scFv that may specifically recognize tumor-associated antigen can couple with a T cell intracellular activation and proliferation signal domain through a transmembrane region. CAR-expressing T cells bind to tumor antigens in an antigen-dependent, but non MHC-restricted manner to initiate and activate tumor-specific killing responses. The effective activation of CAR-T cells depends heavily on the specificity of antibodies that recognize tumor-associated antigens and the binding affinity. In the current situation where the design of CAR-T cell intracellular signal transduction regions has matured, the design of antigen-binding regions has become the focus and key of the development of new CAR-T technologies, wherein the focus is to prevent off-target. The primary risk in the use of CAR-T lymphocytes is off-target effects in clinical application, which can lead to immune responses against normal tissues or cells. As there are few or no known tumor-specific antigens at present, most CAR target tumor-associated antigens that are not expressed or are rarely expressed in important tissues. Therefore, how to improve the targeting of CAR-T lymphocytes is the primary problem, in the clinical application.

The activation of CAR-T cells and the effective killing to target cells depend on the affinity of antibodies that recognize and bind tumor-associated antigens. The CAR expressed on the T cell membrane specifically binds the tumor antigens on the surface of the tumor cell membrane by the frontmost scfv antigen recognition region, so that the tumor cells and CAR-T cells will physically contact, the CAR structure deforms and the T cell activation and killing signals are transferred into T cells. During this period, the surface of CAR-T cells forms a synaptic-like immune synapse structure packaging some tumor cells. The size and intensity of immune synapses are directly related to the affinity of CAR receptors to tumor-associated antigens. The greater the affinity of CAR to tumor-associated antigens is, the larger the immune synapse is. CAR-T cells and tumor cells form immune synapses. At the same time, the conformational transformation of CAR structures also may cause the transmission of T cell activation signals and killing effect signals, and induce T cells to release the perforin and granzyme, thereby causing transcription, expression and secretion of other effectors. The correlation analysis on functions of different near-membrane regions, different tumor antigen targets, and different co-stimulatory signal regions in the CAR structure shows that the affinity of the antigen-binding region to tumor-associated antigens directly affects the conformational transformation of the CAR structure and the signals transmitting ability to T cells, that is, the greater the affinity between the two, the stronger the T-cell activation and killing effect signals are transmitted to CAR-T cells. In addition, considering the dynamic equilibrium relationship between antibodies and antigens, the higher the affinity of antibodies to antigens is, the lower the dissociation probability between them in a unit time is. Macroscopically, this means CAR-T cells will not easily dissociate and allow tumor cells to escape once they recognize and bind tumor cells bearing correct tumor-associated antigens. In summary, for tumor antigens with high specificity or antigens such as CD19 and CD20 that are expressed only on mature B lymphocytes, the constructed CAR-T cells on the basis of higher affinity antibodies may obtain high killing efficiency and excellent clinical treatment effect.

For CAR-T technology and products developers, how to enhance the sustainability of CAR-T cells in patients is one of the keys to the long-term therapeutic effect of CAR-T. The research and development of CAR-T is mainly based on the development of targets and the enhancement of the killing activity of CAR-T. The technical changes from the first generation CAR-T to the fourth generation CAR-T are embodied in changes in intracellular signal regions and co-stimulatory molecules of CAR-T. The earliest CAR used only the tyrosine sequence of the CD3ζ signal chain as the co-stimulatory signal to activate T cells. Although the CAR-T cells can be activated and targetedly clear the cells, unfortunately, this can not promote continuous cell proliferation and secretion of IL-2, causing that T cells died very quickly in vivo. The persistence became one major obstacle in the clinical application of the first generation of CAR T cells. In order to enhance the activity, the second-generation CAR-T cells added a co-stimulatory signal CD28 or CD137 on the basis of Signal 1 as “Signal 2”. Signal 2 not only promotes the division of T cells, the synthesis and expression of IL-2, and the secretion of the anti-apoptotic protein Bcl-xL, but also offset the adverse effects from the tumor cell microenvironment, furthermore does not affect the antigen specificity. Different studies have shown that CAR-T cells carrying Signal 2 exhibit superior efficacy and persistence compared with the first generation CAR-T cells. The third generation of CAR-T added another co-stimulatory signal molecule on the basis of Signal 1 and Signal 2 to further enhance the activity. In addition to adding co-stimulatory signal molecules in the intracellular signal region to enhance the cell activity, the researchers have also used other methods to subtly modify CAR so that the modified T cells can exert the greatest therapeutic effect in the body. During fighting against tumor cells, CAR-T's immune attack is often weakened due to the immunosuppressive signals produced by tumors. These signals include inhibitory cytokines IL-4, IL-10, and tumor growth factor β (TGF-β), which can be produced by cells or matrix components from the tumor microenvironment. In one report from Molecular Therapy, the researchers further modified CAR-T cells targeting prostate stem cell antigens (PSCA, a protein, that is highly expressed in prostate cancer cells but not expressed in normal cells), the binding of the extracellular segment of IL-4 receptors with the intracellular segment of IL-7 receptors has caused the CAR-T cells to produce a new type of inverted cytokine receptor (ICR). The studies have shown that, these ICR-expressing T cells have increased the proliferation ability under the stimulation of IL-4 in a pancreatic cancer cell model producing IL-4. Throughout first to fourth generation of CAR-T structures and their clinical efficacy, it can be found that CAR-T cells transmit activation, killing and proliferation signals into inside of T cells by recognizing and binding tumor cell antigens, during this period, CAR-T cells exert the function of killing tumor cells, after that most cells undergo depletion and apoptosis, only a small part of CAR-T cells can be transformed into memory CAR-T cells, these memory CAR-T cells are directly related to long-term efficacy in patients. Therefore, during the design and treatment of CAR structures, the structures and mechanisms that can promote the formation of long-term memory T cells can help patients to obtain long-term benefits. For example, due to the use of CD137 co-stimulatory signals, the intensity of CAR-T cells is reduced when they receive tumor antigens to stimulate proliferation and killing, thereby delaying the time of CAR-T cell exhaustion and promoting the production of more memory T cells. A more effective strategy is to artificially design a low-affinity CAR structure for specific or non-specific antigens when designing the CAR structure, and continue to give CAR-T cells a very weak signal simulation to promote survival and proliferation of CAR-T cells and differentiation into memory CAR-T cells.

The scfv sequence derived from mouse-derived monoclonal antibodies in CAR structures will cause severe human anti-mouse antibody response (HAMA) after being returned to tumor patients, which seriously restricts the safety and clinical efficacy of therapeutic drugs derived from mouse-derived antibodies. Due to historical reasons, the companies developing CAR-T products such as CD19 CAR-T globally use mostly mouse-derived FMC63 monoclonal antibody strains. Mouse-derived antibodies can induce rejection response in the human body, which severely reduces the persistence of CAR-T cells in patients and will ultimately affect the clinical efficacy and faster relapse. For example, Novartis' Kymriah is a CAR-T product that has been on the market, wherein FMC63 was as the antigen recognition domain, and 10% of patients relapse within 6 months after treatment, and about 45% of patients relapse after treatment of 12 months. One important reason is the immunological rejection induced by mouse-derived components introduced by CAR-T cells. Humanization of mouse-derived antibodies by genetic engineering or human-derived antibodies by directly screening human antibody libraries and reconstruction of human-derived CAR-T theoretically helps to reduce or avoid the immune response in patients to CAR-T cells, maintain long-term presence of CAR-T cells in patients and improve long-term treatment effect.

Multiple myeloma (MM) is a disease caused by the clonal proliferation of malignant plasma cells in bone marrow and peripheral blood, which causes bone marrow hematopoietic inhibition and osteolytic symptoms. Although the application of traditional medicines and hematopoietic stem cell transplantation and targeted drugs (such as chemotherapeutic drugs, proteasome inhibitor drugs and immunomodulatory drugs) can achieve a very good clinical result, most patients develop drug resistance or relapse after a certain period of treatment. At the present stage, MM still belongs to a class of malignant diseases that cannot be clinically cured. One of the important reasons is immune deficiency and immune tolerance with the development of the diseases. Therefore, immunotherapy is likely to become a clinical method for curing MM in the future. In innate immune systems, the cytotoxicity and immunoregulatory functions of NK cells in MM patients are weakened, tumor-associated macrophages are activated, and a large number of pro-inflammatory cytokines such as TNF-α and IL-6 are secreted to promote the growth of MM cells. The ability of dendritic cells in MM patients to phagocytose bacteria and present antigens reduces, and thereby they cannot effectively utilize tumor antigens to stimulate and activate T cells to exert antitumor effects. In acquired immune systems, the functions of T cells and B cells in MM patients are weakened. MM cells and MM-derived bone marrow stromal cells promote the shift of Treg/Th17 balance to Th17. Th17 cells have immunosuppressive functions and can promote tumor growth. Both the innate immune systems and the acquired immune systems produce immune tolerance to MM. Therefore, the occurrence and development of MM cannot effectively be fighted depending only on the patient's own immune regulation, and even if in the growth of MM cells under the conditions of external drugs, the regulatory effect of the immune systems is still insufficient to completely control the progression of tumors, so the role and function of the patient's own immune system must be considered in current treatment strategies.

CAR-genetically modified autologous T cells recombine tumor antigen-specific recognition antibodies or domains with co-stimulatory signals that stimulate T cell activation and proliferation, enabling them to simulate killing effect of cytotoxic T cells or effector T cell to tumor cells in vitro and in vivo. The most important is that such genetically modified CAR-T cells have MHC-independent tumor antigen recognition and killing ability, and can specifically proliferate under the stimulation of specific antigens. After such CAR-T cells are returned to patients, some cells may be transformed into specific memory T cells and survive in patients for a long time while exerting specific killing functions. When the memory T cells are stimulated again by tumor antigens, these cells will rapidly proliferate and kill new tumor cells, thus patients can get long-term clinical remissions and even cures. In recent years, CAR-T therapy targeting malignant hematological tumors derived from B lymphocytes has got great clinical success. For example, refractory and relapsed acute B lymphocytic leukemia in clinical may achieve a complete remission rate of 90%, refractory and relapsed B-lymphocyte-derived lymphoma may also achieve a complete remission rate of 50% by CAR-T targeting CD19, CD20, CD22 and other targets, this fully illustrates that CAR-T therapy is promising in the treatment of malignant tumors. At the present stage, a number of domestic and foreign research institutes and drug development companies have completed successively the preclinical development of chimeric antigen receptor T cells targeting MM specific antigens and entered clinical research and achieved a milestone in treatment effects. In a report from the 2017 Annual Meeting of the American Society of Clinical Oncology, Nanjing Legendary Biotherapy Company from China reported a LCAR-B38M-CAR-T therapy targeting CD269 (B-Cell Maturation Antigen, BCMA), there were 35 patients relapsed or treatment-resistant multiple myeloma participated in this clinical study, and the objective remission rate of the therapy reached 100%. Among the 19 patients who were firstly treated, 14 achieved strict complete remission (sCR), and the remaining 5 patients achieved partial remission, in which 5 patients who had been treated for more than 1 year were still in the sCR stage; CAR-T therapy bb2121 from Bluebird Bio. Company also targets CD269 protein. 15 patients treated with 3 different doses of bb2121 showed an objective response, in which 4 patients achieved a complete response. Another 3 patients were treated with a fourth lower dose of bb2121, all of them died. If these 3 patients are also considered, the overall response rate of bb2121 is 89%. These successes from clinical studies bring the treatment of MM into a new era of immunotherapy. In the future, CAR-T therapy targeting MM-specific antigens is very promising to be a cure for MM.

BCMA (B Cell Maturation Antigen, CD269) is slightly expressed in mature B cells and plasma cells, and its expression is significantly up-regulated in MM, it is a reliable indicator for the diagnosis, and development of MM in clinical. At the same time, BCMA protein is only expressed in mature B cells or plasma cells, and not expressed in memory B cells and naive B cells and other tissues. Therefore, BCMA is a very ideal target for immunotherapy such as CAR-T or antibody drug therapy. ADC drugs (GSK2857916) and dual-targeting (T cells and tumor cells) antibody drugs (BI836909) targeting BCMA are now in the Phase I clinical study and preclinical research. The BCMA CAR-T products targeting BCMA from University of Pennsylvania, National Cancer Institute, and Bluebird Bio have entered Phase I clinical trials. The CAR-T products targeting BCMA form domestic Nanjing Legend Biotechnology Co., Ltd. have entered the research phase of Phase I clinical trial. The BCMA CAR-T products from the above research institutes or drug development companies specifically target the BCMA protein on the MM cell membrane, but the BCMA protein expressed on the MM cell membrane can be cleaved by proteases and enter the blood circulation under physiological conditions, especially the level of soluble BCMA protein in the serum of MM patients will increase, and the increased level is positively correlated with the tumor malignancy. Free BCMA in serum can be combined with CAR-T cells targeting BCMA, which can directly cause the exhaustion of CAR-T cells or reduce the anti-tumor effect of CAR-T cells targeting BCMA. Therefore, there is an urgent need to develop an improved or jointly BCMA-targeted CAR-T technology aiming to achieve better clinical treatment results.

CD38 is a glycoprotein located on the membrane, which catalyzes the synthesis and degradation of cyclic ADP-Ribose (cADPR). The expression and distribution of CD38 molecule is quite broad without limiting cell lines, and the progenitor cells demonstrate high levels of expression in directed myeloid and lymphocyte lines and also have a certain levels of expression in NK cells, T cells, B cells, etc. Compared with normal plasma cells, the expression of CD38 on the surface of myeloma cells is significantly increased, and the activation of CD38 on the cell surface promotes the phosphorylation of cell substrates to activate the NF-κB signaling pathway, while the NF-κB signaling pathway is closely related with drug resistance in MM cells, these evidences suggest that CD38 is a potential target for treating multiple myeloma. In 2015, FDA granted accelerated approval to the monoclonal antibody drug Darzalex (Daratumumab) targeting CD38 for the treatment of MM. Daratumumab has broad-spectrum killing activity and targets highly expressed transmembrane extracellular enzyme CD38 on the surface of MM cells. It can induce rapid apoptosis of tumor cells through a variety of mechanisms, prolong patients' survival time, and will not seriously inhibit the growth of myeloid cells. CD38-targeted CAR-T cells (CD38CAR-T cells) specifically kill cells in MM cell lines and primary MM cells in vitro, but there is no report on the clinical data of CD38 CAR-T so far.

In addition, traditional B cell markers such as CD19 are not favored by traditional MM immunotherapy, however, a case of MM successfully treated by CD19 CAR-T reported by U Penn and Novartis on NEJM last year seems to bring new hope for such type of antigens. The article pointed out that a group of MM tumor cells with strong drug resistance and strong proliferation ability (with the characteristics of tumor stem cells) had low CD19 expression, but indeed CD19 positive. In this case, although 99.5% of malignant hyperplasia plasma cells lack the CD19 expression, the patient was still fully cured. As the patient accepted autologous hematopoietic stem cell transplantation, the effectiveness of CAR-T cell therapy does not exclude the role of ASCT. If CD19 CAR-T proves effective in more patients in future MM clinical trials, this will be a big breakthrough in MM treatment. In addition, the tumor antigens such as cell membrane surface glycoprotein (CS1 or signalling lymphocytic activation molecule 7, SLAM7), immunoglobulin light chain, Lewis Y antigen, esophageal squamous cell carcinoma antigen (New York Esophageal-1, NY-ESO-1), CD44 isomer 6 (CD44v6) and CD138 also have high detection rate in MM cell lines or patient samples. In the future, antibody drugs or CAR-T products targeting these sites also will have a positive effect for treatment of MM.

### SUMMARY OF THE INVENTION

Thereby, in order to solve the above problem, the present invention provides a bispecific chimeric antigen receptor modified T lymphocytes.

The above object of the present invention is achieved by the following technical solution:

A first aspect of the present invention provides a chimeric antigen receptor, consisting of a signal peptide, two specific antigen-binding fragments, an extracellular spacer region, a transmembrane region, an intracellular co-stimulatory signaling domain, the first antigen that is recognized and bound by the specific antigen-binding fragments is a member selected from the group consisting of CD19, CD20, CD22, CD33, CD269, CD138, CD79a, CD79b, CD23, ROR1, CD30, B cell surface antibody light chain, CD44, CD123, Lewis Y, CD7and CD46; the second antigen that is recognized and bound by the specific antigen-binding fragments is CD38, the two specific antigen-binding fragments is linked by a linker peptide.

Preferably, the chimeric antigen receptor is formed by a cell membrane localization signal peptide, a CD269 specific antigen-binding fragment, a linker peptide, a CD38 specific antigen-binding fragment, an extracellular spacer region, a transmembrane region, an intracellular signaling region and a co-stimulatory domain in a serially connected manner.

More preferably, the CD269 specific antigen-binding fragment scfv comprises a framework region and a complementarity determining region CDR1-3, the amino acid sequence of the CD269 specific antigen-binding fragment scFv is an amino acid sequence shown in SEQ ID NO:1-SEQ ID NO:90 in the sequence list. The CD38 specific antigen-binding fragment scfv comprises a framework region and a complementarity determining region CDR1-3, the amino acid sequence of the CD38 specific antigen-binding fragment scFv an amino acid sequence shown in SEQ ID NO:91-SEQ ID NO:180 in the sequence list.

Even more preferably, the present invention screens human monoclonal antibodies targeting CD269 and CD38 with different affinities, so that single-chain antibodies targeting CD38 have a lower affinity for targeting proteins, and single-chain antibodies targeting CD269 have a high affinity for targeting proteins, in particular, the binding affinity constant Kd of the CD269 specific antigen-binding fragment to CD269 is <5.4×10⁻⁸M. The binding affinity constant Kd of the CD38 specific antigen-binding fragment to CD38 is between 5.2×10⁻⁷M and 4.2×10⁻⁵M. In the present invention, due to the specific affinity combination, the low affinity has the ability to quickly bind and release antigens, can mainly enhance the ability of the BCMA recognition region to capture antigens and avoid insufficient affinity under the condition of low-density antigens, and low-affinity antigens per se does not have a killing effect.

More preferably, the linker peptide is (GGGGS)n or (EAAAK)n, wherein 1≤n<4. They represent flexible linker peptides and rigid linker peptides respectively. The study has found that the rigid linker peptides have better killing effect, the reason is that both sites are bound, larger structural deformation are caused by the rigid linker peptides, thereby inducing T cells to have a stronger specific killing effect. Still more preferably, the linker peptide is EAAAK.

More preferably, the cell membrane localization signal peptide of the chimeric antigen receptor is a membrane localization signal peptide of the cell membrane localized proteins selected from the group consisting of CD4, CD8, G-CSFR and GM-CSFR; in order to better express membrane proteins in T cells, signal peptides from highly expressed proteins in T cells are selected. Still more preferably, the cell membrane localization signal peptide includes membrane localization signal peptides of CD8, GM-CSFR.

More preferably, the extracellular spacer region of the chimeric antigen receptor is an extracellular domain of proteins selected from the group consisting of CD4, CD8, CD28, CD137, CD27, PD-1, OX40, TLR4, ICAM-1, ICOS(CD278), NKp80(KLRF1), NKp44, NKp30 and NKp46. The present invention preferably uses the transmembrane region of native proteins in T cells.

More preferably, the transmembrane region of the chimeric antigen receptor is a transmembrane domain of proteins selected from the group consisting of CD4, CD8, CD28, CD137, CD27, PD-1, IL2Rβ, IL2Rγ, IL7Rα, NKG2D, NKG2C, IgG4 and IgG1. The present invention preferably uses the transmembrane region of native proteins in T cells, target CD38 is located inside the CAR domain, and short domain in the extracellular region can be used. As the domain is compact, the induced T cell deformation is greater, and the killing response is stronger after binding antigens.

More preferably, the intracellular signaling region of the chimeric antigen receptor is an intracellular domain of proteins selected from the group consisting of CD2, CD3ζ, CD7, CD27, CD28, CD137, CD134, LCK, TNFR-1, TNFR-2, Fas, NKG-2D, DAP10, DAP12, B7-H3, TLR2 and TLR4IL7R or any combination thereof; preferably, the intracellular signaling region comprises the combination of a CD137 intracellular domain and a CD3ζ intracellular domain, which has mild stimulation to CAR-T cells and better sustainable proliferation ability.

More preferably, the chimeric antigen receptor contains: (1) an amino acid sequence of any light chain variable region listed in SEQ ID NO:1-SEQ ID NO:180 in the sequence list; or (2) an amino acid sequence of any light chain variable region listed in SEQ ID NO:1-SEQ ID NO:180 in the sequence list, in which at least 1 but not more than 30 amino acid sequences are modified; or (3) an amino acid sequence having 90-99% identity to the amino acid sequence of any light chain variable region listed in SEQ ID NO:1-SEQ ID NO:180 in the sequence list.

Alternatively, more preferably, the chimeric antigen receptor contains: (1) an amino acid sequence of any heavy chain variable region listed in SEQ ID NO:1-SEQ ID NO:180 in the sequence list; or (2) an amino acid sequence of any heavy chain variable region listed in SEQ ID NO:1-SEQ ID NO:180 in the sequence list, in which at least 1 but not more than 30 amino acid sequences are modified; or (3) an amino acid sequence having 90-99% identity to the amino acid sequence of any heavy chain variable region listed in SEQ ID NO:1-SEQ ID NO:180 in the sequence list.

Alternatively, more preferably, the chimeric antigen receptor has an amino acid sequence depicted by different constructs in the Table 4. The amino acid sequence of the chimeric antigen receptor is coded by nucleotide codons described in Table 1.

The present invention also provides a method for preparing the bispecific chimeric antigen receptor modified T lymphocytes. The preparation process is briefly described as follows: (1) synthesizing a sequence of CAR domain to construct a lentivirus expression vector; (2) preparing a lentivirus packaging vector by plasmid extraction; (3) transfecting HEK-293T cells by a packaging vector; (4) collecting and purifying the lentivirus; (5) isolating and activating T cells, transducting virus and expanding cells.

A second aspect of the present invention provides a polypeptide, the polypeptide codes the bispecific chimeric antigen receptor (the dual-targeting specific chimeric antigen receptor).

A third aspect of the present invention provides a gene, the gene codes the bispecific chimeric antigen receptor (the dual-targeting specific chimeric antigen receptor).

A fourth aspect of the present invention provides a genetically-engineered virus, the virus may express the bispecific chimeric antigen receptor (the dual-targeting specific chimeric antigen receptor) in a host cell.

A fifth aspect of the present invention provides a genetically-engineered effector cell, the effector cell expresses the polypeptide sequence of the bispecific chimeric antigen receptor, the effector cell is a member selected from the group consisting of T lymphocytes, NK cells, hematopoietic stem cells, pluripotent stem cells, embryonic stem cells and induced pluripotent stem cells, or T lymphocytes and NK cells which is formed by inducing, culturing and differentiating the stem cells. Preferably, autologous or allogeneic T lymphocytes are used.

Preferably, in the genetically-engineered effector cell, the bispecific chimeric antigen receptor expresses on the cell membrane, and may specifically bind the corresponding antigen.

A sixth aspect of the present invention provides a use of the chimeric antigen receptor in the preparation of antitumor drugs, anti-autoimmune disease drugs or anti-viral infectious disease drugs. Preferably, the chimeric antigen receptor is used in the preparation of anti-malignant tumor drugs in the blood system.

Compared with the prior arts, the preparation method of the present invention has the following advantages:

The bispecific chimeric antigen receptor modified T lymphocytes according to the present invention may recognize specifically and kill tumor cells, which express both CD269 and CD38 antigens, and have very strong specificity. The present invention constructs low affinity of chimeric antigen receptors and high affinity of chimeric antigen receptors, which recognize respectively two kinds of tumor-associated antigens, when being transfected into T lymphocytes, the modified T lymphocytes may recognize simultaneously two kinds of tumor-associated antigens and are effectively activated, thereby enhancing the targeting of CAR-T cells to kill tumors and the persistence of CAR-T cells in patients, and preventing the patients from recurring within a short period after treatment with CAR-T.

### BRIEF DESCRIPTION OF THE DRAWINGS

FIG. 1 shows the structures of BCMA & CD38 chimeric antigen receptors.

FIG. 2 shows the binding of BCMA scFv to recombinant antigen BCMA.

FIG. 3 shows the screening of BCMA monoclonal antibody.

FIG. 4 shows the expression and purification of BCMA recombinant antibody.

FIG. 5 shows the binding activity of recombinant antibodies to BCMA on the surface of K562-BCMA cells by FACS analysis.

FIG. 6 shows the screening of CD38 monoclonal antibody.

FIG. 7 shows the binding of CD38 scFv to CD38 recombinant antigen.

FIG. 8 shows a frame flow of CAR recombinant lentiviral.

FIG. 9 shows the expression of the CAR structure during the detection of T cells by BCMA-Fc recombinant proteins

FIG. 10 shows in vitro killing activity of BM38CAR-T cells.

FIG. 11 shows the lifetime of tumor-bearing mice into which BM38 CAR-T are reinfused back.

FIG. 12 shows the flow analysis of BCMA-K562 and CAR-T cells in the peripheral blood and bone marrow from T cells group. FIG. 13 and FIG. 14 show FCS analysis of BCMA-K562 and CAR-T cells in the peripheral blood and bone marrow from BCMA CAR-T group. FIG. 15 shows FCS analysis of BCMA-K562 and CAR-T cells in the peripheral blood and bone marrow from BM38 CAR-T group.

### BRIEF DESCRIPTION OF THE TABLES

Table 1 shows the amino acid codons.

Table 2 shows the binding affinity of recombinant BCMA monoclonal antibody to rBCMA.

Table 3 shows the binding affinity of recombinant CD38 monoclonal antibody to rCD38.

Table 4 shows different CAR constructs.

Table 5 shows the components within the qPCRmix Master I.

Table 6 shows the components within the qPCRmix Master II.

Table 7 shows the titer detection of recombinant lentivirus.

Table 8 shows the titer of Jurkar cells infected with BCMA-Fc recombinant lentivirus.

Table 9 shows in vitro killing efficiency of BM38CAR-T.

### DETAILED DESCRIPTION OF THE INVENTION

Hereafter the present invention will be described in more detail with reference to the accompanying drawings and embodiments. The following examples are merely exemplary, and are only used to further describe the technical solution of the present invention. Those skilled in the art should understand that various modifications or replacements without departing from the spirit and scope of the present invention should be covered within the protection scope of the present invention. The experimental methods without the specific experimental conditions are usually in accordance with the conventional conditions or in accordance with the recommended conditions by manufacturers.

The present invention provides a human single chain antibody specifically targeting BCMA and CD38. In the following embodiments, the antibody of the present invention is derived from specific heavy and light chain sequences, and/or contains specific structural characteristics, for example contains a CDR region and a framework region of specific amino acid sequences. The present invention provides a method for screening and preparing antibodies, identifies the binding characteristics of the above antibodies by flow cytometry, constructs a CAR structure, and provides a preparation and application method of CAR-T cells carrying the corresponding CAR molecules.

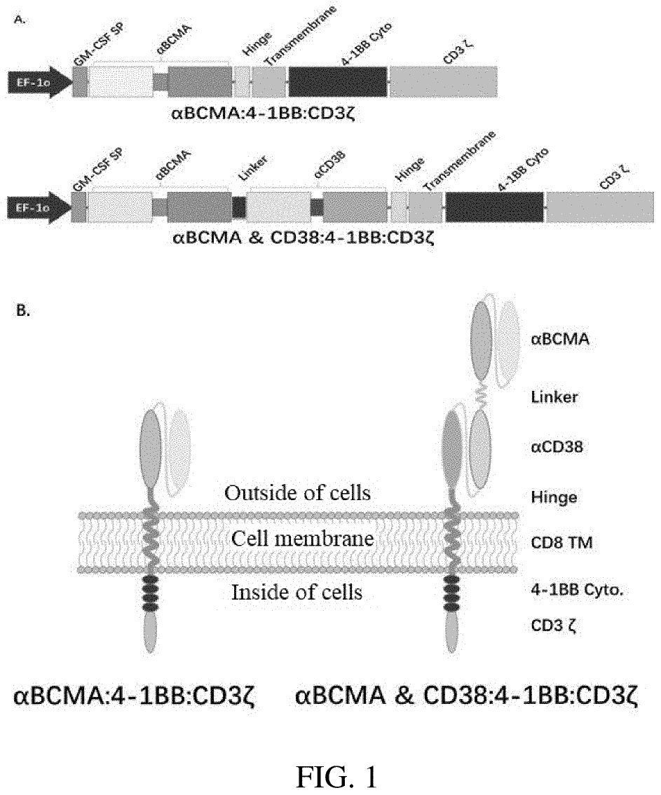

In various embodiments of the present invention, at least one chimeric antigen receptor was provided, its structure and distribution were shown in FIG. 1. The chimeric antigen receptor was spliced sequentially by the specific antigen-binding fragment targeting CD269, the linker peptide, the specific antigen-binding fragment targeting CD38, the CD8α near-membrane region and the transmembrane region, 4-1BB intracellular domain and intracellular domain of CD3ζ chain from the amino-terminus to the carboxy-terminus. The structure of the obtained chimeric antigen receptor CAR was CD269 (scFv) -CD38 (scFv) -CD8α-CD3ζ. Research results showed the order of dual targets had a greater effect of the resulting chimeric antigen receptor CAR. The chimeric antigen receptor was expressed on the membrane of T lymphocytes by genetic engineering, the T cells can recognize and bind specifically the CD269 and CD38 extracellular domains on the surface of target cells at the same time. Wherein, the single-chain antibodies targeting CD38 had slightly lower affinity to the target protein CD38 with a KD value between 5.2×10⁻⁷ M and 4.2×10⁻⁵ M.

### EXAMPLE 1

### Construction of Human Stable BCMA-Expressing Cell Lines

1.1 Construction of a Plasmid Vector

The vector system used in this example belongs to the third-generation, self-inactivating lentiviral vector system, this system has three plasmids, i.e., a packaging plasmid psPAX2 for coding the protein Gag/Pol and coding the protein Rev, an envelope plasmid pMD2.G for coding the protein VSV-G and a recombinant plasmid pCDH-CMV-huCD19-EF1-GFP-T2A-Puro for coding human BCMA extracellular region and transmembrane region based on an empty vector plasmid pCDH-CMV-MCS-EF1-GFP-T2A-Puro (purchased from Addgene company).

In accordance with the human BCMA sequence provided by Genbank Accession No. NM_001192.2, a PCR-based gene synthesis was used for synthesizing a signal peptide, a human BCMA extracellular region, a transmembrane region and an intracellular region, PCR amplification of

SEQ ID NO: 187
(huBCMA-F): 5>ATGTTGCAGATGGCTGGGCAG<3
SEQ ID NO: 188
(huBCMA-F): 5>TACCTAGCAGAAATTGATTTC<3

was performed with primers, the amplification conditions were as follows: pre-denaturation: 94° C., 4 min, denaturation: 94° C., 30 s; annealing: 58° C., 30 s; extension: 68° C., 80 s; 30 cycles. The theoretical size of the obtained fragment was 1716 bp, the amplified product had the same size as the theoretical size by analyzing using agarose gel electrophoresis. Wherein, the XhoI and BamHI restriction sites were introduced into the upstream and downstream of the open reading frame. The obtained targeted gene was double digested by XhoI and BamHI, and linked into a pCDH-CMV-MCS-EF1-GFP-T2A-Puro vector digested by the same double enzymes. The successfully constructed lentiviral vector pCDH-CMV-huBCMA-EF1-GFP-T2A-Puro was packaged after identification by XhoI and BamHI enzymes digestion and sequencing without errors.

1.2 293 Plasmid Transfected T Cells Packaging Lentiviral

293 T cells (ATCC: CRL-11268) was inoculated at a density of 6×10⁶ to the 6th˜10th generations in a 10 cm petri dish, incubated overnight at 37° C. with 5% CO₂, which is ready for transfection. The medium is a DMEM (ThermoFisher company) containing 10% phage-free fetal bovine serum (Hangzhou Sijiqing company), the next day, the medium was changed into serum-free DMEM about 2 hours prior to transfection.

The transfection steps were as follows:
1) 5 μg of target gene plasmid pCDH-CMV-huBCMA-EF1-GFP-T2A-Puro, 7.5 μg of packaging plasmid pCDH-CMV-huBCMA-EF1-GFP-T2A-Puro, pSPAX 2 and 2.5 μg of envelope plasmid pMD2.G were dissolved in a 500 μL Mill Q water and mixed well;
2) 62 μL of 2.5 M CaCl₂ (Sigma Corporation) was added dropwise and vortex mixed at 1200 rpm/min;
3) finally, 500 μL of 2× HBS (280 mM NaCl, 10 mM KCl, 1.5 mM Na₂HPO₄, 12 mM glucose, 50 mm Hepes (Sigma Corporation), pH 7.05, filter sterilization by 0.22 μM) was added drop wise and mixed by shaking at 1200 rpm/min for 10 s;
4) the formation was added immediately drop wise into a petri dish, and shaked gently at 37° C. with 5% CO₂, the medium was changed into DMEM containing 10% fetal bovine serum after 4-6 h incubation.

After 48 h or 72 h transfection, the cell debris was removed by centrifugation, followed by the virus was collected by filtration through a 0.45 μm filter (Millipore Corporation).

1.3 Recombinant Lentiviral Infected Leukemia Hela or K562 Cells

After the collected virus solution was concentrated and titrated, and was used to infect the Hela or K562 cells in a 6-well plate. Three days after infection, the cells were collected, part of the cells was taken and the cell-surface BCMA expression was detected by flow cytometry. The remaining cells were incubated for expansion, and some of the cells was frozen and saved, others were passaged in 6-well plate, 2 μg/ml of Puromycin antibiotic (purchased from Sigma) was added to screen for 1-2 weeks till all cells in view expressed GFP under microscope observation or the BCMA expression was detected by flow cytometry, when compared with the negative cells, the stably transfected cells should exhibit positive GFP and BCMA expression. The frozen cells was used for the killing assay by subsequent flow cytometry, the amount of the antibiotic was halved during subsequent cultivation to maintain over-expression of the corresponding genes.

### EXAMPLE 2

### Screening and Identification of Specific Single-Chain Antibody (scFv) Binding to Human BCMA

2.1 Screening of Anti-Human BCMA Specific Single Chain Antibody by Phage Display

The sequence of the single-chain antibody specifically binding to human BCMA was screened from directionally modified human single-chain antibody phage libraries by phage display technology of single chain antibody.

To achieve such objective, a glycerol bacteria from a phage display natural library (a self-constructing library) of fully human single chain antibodies was inoculated in a 400 ml 2×YT/ampicillin medium to reach a cell density of OD600=0.1, and cultured at 37° C. under shaking condition (200 rpm) till the cell density reached OD600=0.5. 10¹² Pfu of M13KO7 helper phage (purchased from ThermoFisher Corporation) was used to infect and the cultivation was performed for 30 minutes at 30° C. and 50 rpm. After adding 50 mg/L kanamycin, the cultivation was performed for 30 minutes at 37° C. under shaking condition (200 rpm), the precipitate was separated through centrifugation (15 minutes, 1600×g, 4° C.) and resuspended in the 400 ml 2×YT/ampicillin/kanamycin medium, the cultivation was performed for 16 hours at 37° C. under shaking condition (200 rpm). Finally, the precipitate was separated through centrifugation (20 minutes, 5000×g, 4° C.) and discarded, the supernatant was filtered through a 0.45 μm filter, added with ¼ volume of 20% (w/v) PEG8000, 2.5M NaCl solution and incubated for 1 hour in an ice bath to precipitate phage particles. Followed by centrifugation (20 min, 8000×g, 4° C.), the supernatant was discarded, the phage precipitate was resuspended in a 25 ml pre-cooled PBS solution (NaCl, 2.7 mM KCl, 8 mM Na₂HPO₄, 2 mM KH₂PO₄) and centrifuged (5 minutes, 20000×g, 4° C.). 1/4 volume of 20% (w/v) PEG8000, 2.5M NaCl solution was added to the supernatant, and put in an ice bath for 30 minutes to re-precipitate phage particles. Followed by centrifugation and precipitation (30 min, 20000×g, 4° C.), the phage precipitate was resuspended in a 2 ml pre-cooled PBS solution, kept in an ice bath for 30 minutes and centrifuged (30 minutes, 17000×g, 4° C.). The supernatant was mixed with a PBS solution containing 4% (w/v) BSA in a ratio of 1:1, placed on a rotary mixer, maintained at room temperature for 30 minutes, and then used directly for screening.

The directional screening for a biotin-labeled recombinant human Fc-BCMA protein (available from Acrobiosystems Corporation) was implemented for 3 rounds using the phage antibody libraries, the screening protocol was as follows:

The phage antibody libraries and the biotin-labeled recombinant human Fc-BCMA antigen were incubated for 2 hours at room temperature, and then were incubated for 30 minutes at room temperature with streptavidin-labeled Dynabeads® magnetic beads (available from ThermoFisher Corporation) blocked with 2% BSA solution. And then the magnetic beads were washed with PBST (containing 0.1% Tween-20) buffer, the phage having non-specific binding or weak binding ability was removed. The phage having strong binding ability was eluted with glycine-hydrochloric acid buffer (pH 2.2) from the magnetic beads and neutralized with Tris neutralizer (pH 9.1) for infecting E. coli ER2738 in logarithmic growth phase and the next round of screening. During the 3 rounds of screening, the amount of the magnetic beads were 50 μl, 20 μl and 10 μl respectively, the concentration of the biotin-labeled human Fc-BCMA antigen were 100 nM, 10 nM and 1 nM respectively, the numbers of washing with PBST were 10 times, 15 times and 20 times respectively.

1.2 Identification of Human BCMA Specific Single Chain Antibody

Single clone were randomly selected from the third round of screening clones, its binding capacity to human Fc-BCMA was analyzed by single phage ELISA (enzyme-linked immunosorbent assay). For this purpose, each single colony was inoculated with 300 μl 2×YT/ampicillin medium (containing 2% of glucose) in a 96-well deep-well plate, and cultured for 16 hours at 37° C. under shaking condition (250 rpm). 20 μl cultures were inoculated to a 500 μl 2×YT/ampicillin medium (containing 0.1% of glucose), and cultured for 1.5 hours at 37° C. under shaking condition (250 rpm). A helper phage solution was prepared, 75 μl of M13KO7 (the titer was 3×10¹² pfu/ml) was mixed into a 15 ml 2×YT medium, added to the culture plate in 50 μl/well. The cultivation was performed for 30 minutes at 37° C. and 150 rpm, followed by the prepared kanamycin solution (180 μl 50 mg/ml of kanamycin was taken and added to a 15 ml 2×YT medium) was added in 50 μl/well, and the cultivation was performed for 16 hours at 37° C. under shaking condition (250 rpm). Finally, the cells were centrifuged and precipitated (30 minutes, 5000×g, 4° C.), the supernatant was transferred to a new 96-well deep-well plate.

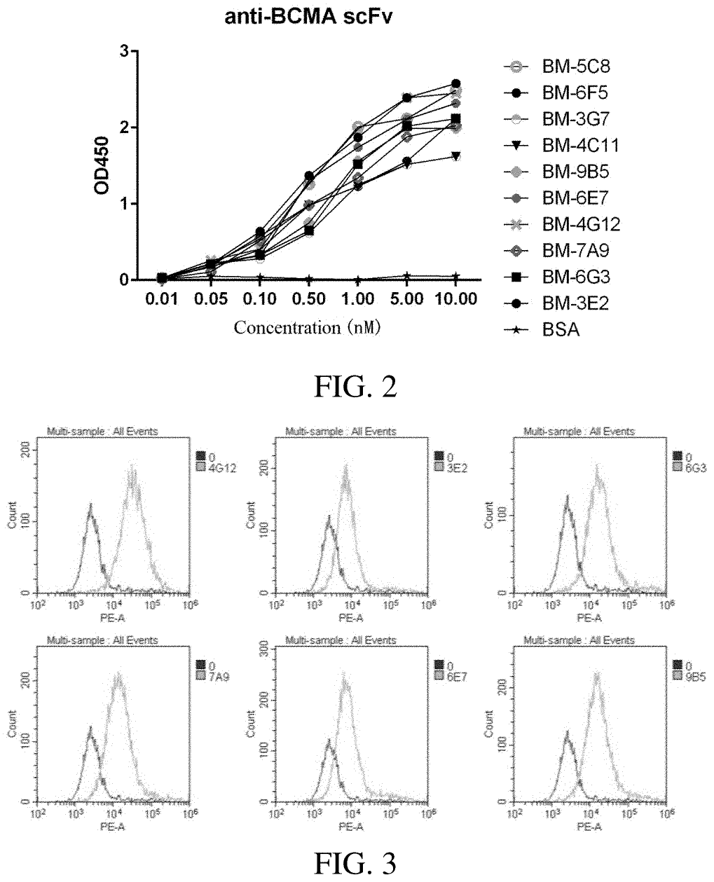

To perform a single phage ELISA, a human Fc-BCMA recombinant antigen (100 ng/well) and a negative control protein Fc (100 μl/well) in a 96 well MediSorp ELISA plate (available from Thermo Fisher) were coated overnight at 4° C. Each well was closed by a PBST solution containing 2% BSA. After that the wells were washed three times with PBST and patted to clean. And then each prepared phage solution was added to each well in the plate in 100 μl/well. Washing was performed with PBST three times after incubation at 37° C. for 2 hours. In order to detect bound phages, anti-M13 antibody superoxide dismutase conjugate (purchased from Proteintech Group, Inc) was diluted in PBST at 1:5000, 100 μl was added to each well. Washing was performed with PBST three times after incubation at 37° C. for 1 hours, and then washing was performed with PBST three times. Finally, 50 μl TMB substrate was drawn and added to wells, and developed for 10 minutes at room temperature, and then 50 μl of 2 M H₂SO₄ per well was added to stop the color reaction. The extinction value was determined by enzyme-linked immunosorbent assay (Bio-Rad) at 450 nm. For the wells which was judged as positive in accordance with the extinction value, the phage plasmid was extracted to perform a sequencing verification for scfv sequences, the amino acid sequence of the obtained single-chain antibodies was analyzed as shown in the sequence list, the single-chain antibodies of the present invention had very strong binding signal to human Fc-BCMA in ELISA experiments, had no binding or very weak binding to Fc recombinant protein, the binding curve of the single chain antibodies to different concentrations of BCMA antigen was shown in FIG. 2.

In order to initially determine whether the phage obtained by screening which express scFv may bind to the BCMA native antigen, the concentrated phage by centrifugation was used to stain and perform a flow analysis for BCMA-K562 cells. The binding ability of the concentrated phage with human BCMA antigen on cell surface was analyzed by a flow cytometry (CytoFLEX, Beckman). The specific method was as follows:

1) Raji, K562-BCMA and K562 cells in logarithmic growth phase were taken, inoculated in a T25 cell culture flask with an inoculation density of about 5×10⁵ cell/ml and cultured overnight at 37° C. in a culture incubator.

2) The culture medium in the petri dish was slightly shaked, the cells were collected by centrifugation at 200 g×5 min, resuspended in a phosphate buffer (NBS PBS) containing 1% of bovine calf serum at a concentration of 2˜3×10⁶/mL and added into a flow special tube in an amount of 100 ul/tube.

3) The centrifugation at 200 g×5 min was performed, the supernatant was discarded.

4) The phage to be tested and positive antibodies were respectively added into two experimental groups, a PBS blank control without antibodies was set as the control group. The final concentration of each phage was 20 μg/ml, each tube was added with 100 ul and placed in an ice bath for 45 minutes.

5) Each tube was added with 2 ml 1% NBS PBS and centrifuged at 200 g×5 min two times.

6) The supernatant was discarded, goat anti-human antibody FITC (ProteintechGroup, Inc.) at a dilution of 1:100 was added at 100 ul/tube and placed in an ice bath for 45 minutes.

7) Each tube was added with 2 ml 1% NBS PBS and centrifuged at 200 g×5 min two times.

8) The supernatant was discarded, resuspended, in a 300 μl 1% NBS PBS, and detected by flow cytometry.

9) Data was analyzed by flow cytometry data analysis software CytoExpert 2.0.

The flow cytometry results show that, compared with the isotype control IgG, BCMA-K562 cells stained with BCMA-targeted single-chain antibody exhibit significantly different fluorescence peak, but there is no obvious difference for human BCMA negative K562 cells, this indicates that the phage obtained by screening which binds BCMA may recognize specifically human BCMA extracellular region, the result of the typical flow analysis was shown in FIG. 3.

### EXAMPLE 3

### Preparation and Activity Analysis of Anti-BCMA Antibodies

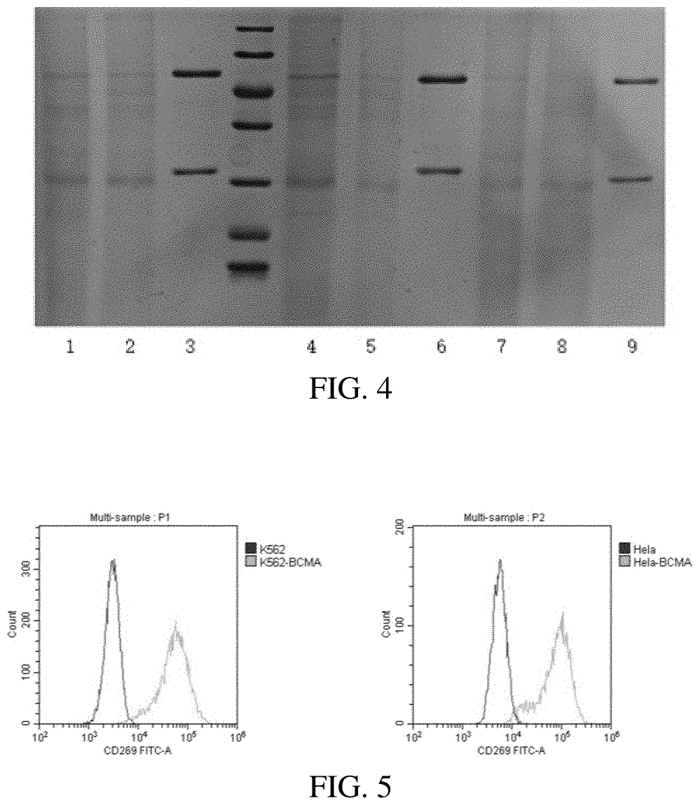

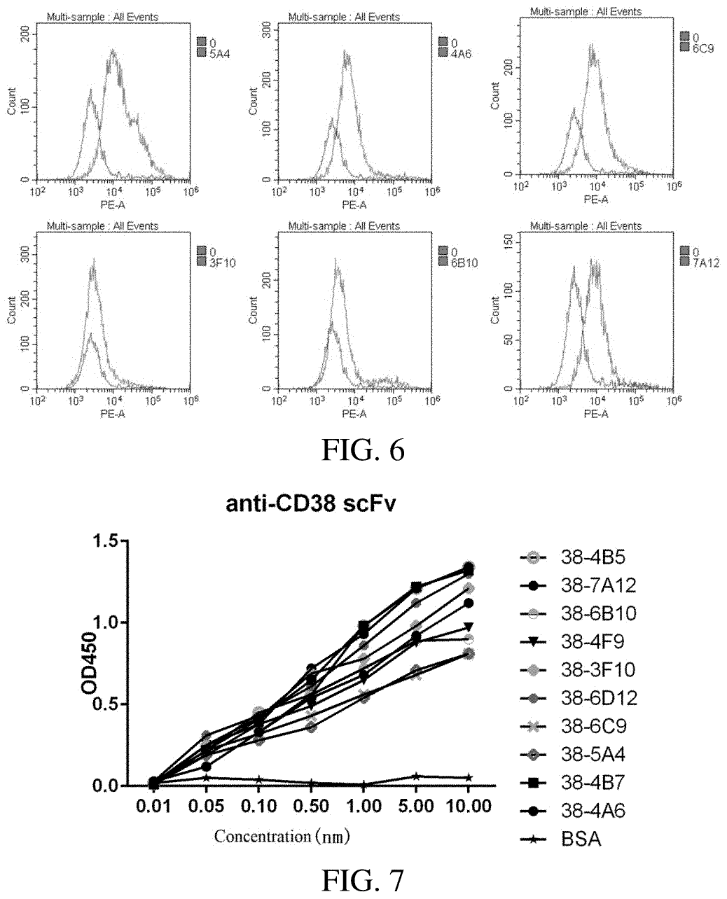

3.1 Light and heavy chain eukaryotic expression vectors of the selected single-chain antibodies were constructed, transfected with HEK293F to induce a recombinant expression and the purification was performed. The light and heavy chains in the sequence of single chain antibody obtained in example 1 were constructed respectively into a monoclonal antibody expression plasmid pCMV-V5-Fc, after the sequence was confirmed by sequencing without errors, the plasmid was prepared in large quantity. The light and heavy chain expression plasmids were mixed in an appropriate ratio, and then transfected into HEK-293 F cells with well growth, and cultured continuously for 7 days at 37° C. with 5% CO₂ in a shaker (125 rpm). Centrifugation at 4000 rpm was performed for 10 min, the precipitate was removed, the supernatant was collected, and washed with 0.45 μm membrane filter, the treated samples were subjected to a Protein A (available from GE Corporation) affinity column to purify, finally, a purified recombinant antibody BCMA with murine IgG Fc regions was obtained, the identification results by polyacrylamide gel electrophoresis was shown as FIG. 4.

FIG. 4 shows the expression detection of BCMA recombinant antibody (wherein, Lane 1: BM-4G12-pcDNA3.4 stock solution; Lane 2: BM-4G12-pcDNA3.4 penetration; Lane 3: BM-4G12-pcDNA3.4 elution peak; Lane 4: BM-9B5-pcDNA3.4 stock solution; Lane 5: BM-9B5-pcDNA3.4 penetration; Lane 6: BM-9B5-pcDNA3.4 elution peak; Lane 7: BM-6E7-pcDNA3.4 stock solution; Lane 8: BM-6E7-pcDNA3.4 penetration; Lane 9: BM-6E7-pcDNA3.4 elution peak.)

3.2 Binding Activity of Recombinant Antibodies to Human BCMA Antigen by ELISA Analysis

The binding activity of the screened antibodies to human BCMA antigen was measured by ELISA experiment in concentration gradient. For this purpose, the human BCMA-His antigen was diluted with a 0.1M NaHCO₃ (pH 9.6) coating solution, each well was coated with 100 ng (50 μl/well) overnight at 4° C. and closed by a PBST blocking solution containing 2% BSA and 0.01% (v/v) Tween-20 for 2 hours at room temperature. PBST was used to wash the plate for three times and removed. After that, each well was added with 100 μl PBST solution containing a series of concentrations (the starting concentration was 10 nM with a 3-fold gradient dilution still 1:729) of each antibody protein. Each sample was measured by parallel three-well analysis. Washing was performed with PBST three times after incubation at 37° C. for 2 hours, and then a horseradish peroxidase-labeled goat anti-human antibody (purchased from Proteintech Group, Inc) with a dilution of 1:20000 was at 100 μl/well, and reacted at 37° C. for 1 hour. In order to detect, the wells were washed three times with PBST, and then washed three times with PBS. Finally, TMB was added to develop for 15 minutes and then 50 μl of 2 M H₂SO₄ per well was added to stop the color reaction. The extinction value was determined by enzyme-linked immunosorbent assay (Bio-Rad) at 450 nm. GraphPad software was used to evaluate the absorbance value, and the binding strength of antibodies was calculated. For this purpose, a plot for the extinction values measured in each case vs the corresponding antibody concentration was drawn, and the resulting curve is fitted by the following nonlinear regression, the apparent affinity was calculated according to the formula A0/(A0−A)=1+KD/a (where A0 is OD value of an antibody in the absence of antigen, A is OD value after adding antigen with a molar concentration of a, and KD is the inverse of the affinity constant), as shown in Table 2. The binding activity of the obtained anti-human BCMA single chain antibody in example 1 to the BCMA-His antigen was between 54 nM and 0.91 nM after being recombinant expressed into a complete antibody.

### EXAMPLE 4

### Construction of Human Stable BCMA-Expressing Cell Lines

4.1 Construction of a Plasmid Vector

The vector system used in this example belongs to the third-generation, self-inactivating lentiviral vector system, this system has three plasmids, i.e., a packaging plasmid psPAX2 for coding the protein Gag/Pol and coding the protein Rev, an envelope plasmid pMD2.G for coding the protein VSV-G and a recombinant plasmid pCDH-CMV-huBCMA-EF1-GFP-Puro for coding human BCMA extracellular region and transmembrane region based on an empty vector plasmid pCDH-CMV-MCS-EF1-GFP-Puro (purchased from Addgene company).

In accordance with the human BCMA sequence provided by Genbank Accession No. NM_001192.2, PCR-based gene synthesis were used for synthesizing signal peptides, a human BCMA extracellular region, a transmembrane region and an intracellular region, PCR amplification of

huBCMA-F(SEQ ID NO 189): 5′-TGTGATCATGTTGCAGAT-3′
huBCMA-R(SEQ ID NO 190): 5′-TACCTAGCAGAAATTGAT-3′

was performed with primers, the amplification conditions were as follows: pre-denaturation: 94° C., 4 min, denaturation: 94° C., 30 s; annealing: 58° C., 30 s; extension: 68° C., 80 s; 30 cycles. The theoretical size of the obtained fragment was 1716 bp, the amplified product had the same size as the theoretical size by analyzing using agarose gel electrophoresis. Wherein, the XhoI and BamHI restriction sites were introduced into the upstream and downstream of the open reading frame. The obtained targeted gene was double digested by XhoI and BamHI, and linked into a pCDH-CMV-MCS-EF1-GFP-Puro vector digested by the same double enzymes. The successfully constructed lentiviral vector pCDH-CMV-huBCMA-EF1-GFP-Puro was packaged after identification by XhoI and BamHI enzymes digestion and sequencing without errors.

4.2 293 Plasmid Transfected T Cells Packaging Lentiviral

293 T cells (ATCC: CRL-11268) was inoculated at a density of 6×10⁶ to the 6th˜10th generations in a 10 cm petri dish, incubated overnight at 37° C. with 5% CO₂, which is ready for transfection. The medium is a DMEM (Thermo Fisher company) containing 10% phage-free fetal bovine serum (Hangzhou Sijiqing company), the next day, the medium was changed into a serum-free DMEM about 2 hours prior to transfection.

The transfection steps were as follows:

1) 5 μg of target gene plasmid pCDH-CMV-huBCMA-EF1-GFP-T2A-Puro, 7.5 μg of packaging plasmid pCDH-CMV-huBCMA-EF1-GFP-T2A-Puro, pSPAX 2 and 2.5 μg of envelope plasmid pMD2.G were dissolved in a 500 μL Mill Q water and mixed well;

2) 62 μl of 2.5 M CaCl₂ (Sigma Corporation) was added dropwise and vortex mixed at 1200 rpm/min;

3) finally, 500 μL of 2× HBS (280 mM NaCl, 10 mM KCl, 1.5 mM Na₂HPO₄, 12 mM glucose, 50 mm Hepes (Sigma Corporation), pH 7.05, filter sterilization by 0.22 μM) was added drop wise and mixed by shaking at 1200 rpm/min for 10 s;

4) the formation was added immediately drop wise into a petri dish, and shaked gently at 37° C. with 5% CO₂, the medium was changed into DMEM containing 10% fetal bovine serum after 4-6 h incubation.

5) After 48 h or 72 h transfection, the cell debris was removed by centrifugation, followed by the virus was collected by filtration through a 0.45 μm filter (Millipore Corporation).

4.3 Recombinant Lentiviral Infected Leukemia Hela or K562 Cells

After the collected virus solution was concentrated and titrated, and was used to infect the Hela or K562 cells in a 6-well plate respectively. Three days after infection, the cells were collected, part of the cells was taken to mix and clone and the cell-surface BCMA expression was detected by flow cytometry and BCMA-targeted fluorescently labeled antibodies. The method and steps by flow cytometry are the same as in Example 5. The remaining cells were incubated for expansion, and some of the cells was frozen and saved, others were passaged in 6-well plate, 2 ug/ml of Puromycin antibiotic (purchased from Sigma) was added to screen for 1-2 weeks till all cells in view expressed GFP under microscope observation. The frozen cells were used for the killing assay by subsequent flow cytometry. For example, the constructed stable BCMA-expressing cell lines K562 and Hela were detected by using the recombinant anti-BCMA monoclonal antibody expressed and purified in Example 2 and the corresponding fluorescently labeled secondary antibody by flow cytometry. The results were shown in FIG. 5, the recombinant expressed antibodies can significantly distinguish BCMA-overexpressing K562 or Hela cells.

### EXAMPLE 5

### Screening and Analysis of Single Chain Antibodies Binding to Targeting CD38

In order to obtain the sequences of monoclonal antibodies binding to targeting CD38, the present example constructed a human CD38 phage library by using the same method as described in Example 2, 10 strains of single-chain antibodies capable of binding specifically to human CD38 were obtained by screening, the CD38 molecules binding to human CD38 cell membrane surface were identified by flow detection.

The binding curve of the recombinant antibodies binding targetedly to CD38 obtained by the present example to CD38 by using recombinant expressed CD38 extracellular region antigen and ELISA method (which is the same as the ELISA method in Example 3) was shown in FIG. 7, wherein the apparent affinity of antibodies targeting CD38 was between 4.2×10⁻⁵ M and 5.2×10⁻⁷ M, the affinity data was shown in Table 3.

### EXAMPLE 6

### Construction of Chimeric Antigen Receptors Targeting BCMA and CD38

The sequences of bispecific chimeric antigen receptors targeting BCMA and CD38 was shown in Table 4, one single chain antibody targeting BCMA and one single chain antibody targeting CD38 were combined, the two single-chain antibodies were linked by a linker peptide, the second single-chain antibody is followed by a transmembrane region and a co-stimulatory signal molecule. As an example, the present example illustrated the construction of dual-targeted chimeric antigen receptors, packaging of recombinant lentivirus, transduction and preparation of CAR-T cells, and detection method of tumor cell killing ability by forming a dual-antigen recognition region by combining one strain of BCMA monoclonal antibody with high affinity and three strains of CD38 monoclonal antibodies with different affinities respectively. In each combination, two scFv fragments were connected respectively in different orders and rigid series, 6 dual-antigen recognition regions (BM38-07, BM38-06, BM38-05, 38BM-05, 38BM-06, 38BM-07) were formed in total, which described the killing ability of the tumors obtained by different combinations and prevention of killing behaviors targeting non-tumor cells.

The experimental materials and equipment used in this example were as follows:

Lentiviral backbone plasmid pLVX-EF1 was constructed by our company and was saved after verification by full-length sequencing without errors, lentiviral packaging plasmid pRSV.REV (Rev expression plasmid), pMDLg/p.RRE (Gag/Pol expression plasmid), pVSV-G (VSV glycoprotein expression plasmid) were purchased from Addgene, HEK293T cells, Jurkat cells were purchased from China Center for Type Culture Collection, LentiX-293 T cells were purchased from Clontech, BCMA-K562 cells was constructed by our company.

Human fresh peripheral blood was provided by healthy volunteers;

0.22 μm-0.8 μm PES filter was purchased from PALL company; 200 mesh cell screen, 10 cm, 15 cm cell culture dishes, 1 L culture bag, 24-well, 6-well culture plates, 10-layer cell culture factory were purchased from Corning company; D-PBS 0.4% of trypan blue was purchased from Thermo company, and polyethylenimine (PEI) was purchased from POLYSCIENCES company.

Opti-MEM, Pen-Srep, Hepes, FBS, AIM-V, RPMI 1640, DMEM, Trypsin, Lipofectamine 3000 were purchased from Thermo company; Biotinylated protein L was purchased from GeneScrip company; LDH detection kit was purchased from Promega company; Ficoll lymphocyte isolation liquid was purchased from GE company; 20% human albumin injection was purchased from CSL Behring company; rIL-2, rIL-7, rIL-15, and rIL-21 were purchased from Cytocares company; CD3 monoclonal antibodies, CD28 monoclonal antibodies. CD3/CD28 magnetic beads and CD4/CD8 magnetic beads were purchased from Miltenyi company;

CD4-FITC and CD8-APC were purchased from BioLegend company, Phycoerythrin (PE)-conjugated streptavidin was purchased from BD Bioscience company; Protein L Magnetic Beads were purchased from BioVision company;

The coding DNA sequences and amino acid combination of CAR constructs BM38-07, BM38-06, BM38-05, 38BM-05, 38BM-06 and 38BM-07 were shown in Table 4. The sequences were synthesized by Wuhan Gene Create Biological Engineering Co., Ltd. The oligonucleotide sequence was loaded into the vector pLVX-EF-1 by restriction endonuclease digestion or homologous recombination, and the resulting recombinant plasmid was saved after verification by sequencing without errors.

### EXAMPLE 7

### Preparation of Dual-Targeted Chimeric Antigen Receptor Recombinant Lentivirus

Preparation of Solution:

DMEM complete medium: DMEM pre-prepared medium stored at 2-8° C. was taken out, added with 10% (v/v) phage-free fetal bovine serum, and stored at 2-8° C. for later use after mixing in upside down;

1× PBS Solution:

0.5M CaCl₂ Solution:

1 g/L of PEI solution: 1 g of PEI powder was weighed, dissolved with 900 ml Milli-Q grade ultrapure water, heated in a water bath to 60-80° C. under continuous stirring, the pH was adjust with HCl to about 2.0 after calibrating the pH meter. The beaker was covered, a continue stirring was performed for 3 h until the powder was completely dissolved. After cooling to room temperature, NaOH was added to adjust the pH to 6.9-7.1. The solution was transferred to a volumetric flask and added with water to dilute to 1 L, filtered through a 0.22 μm PES needle filter and then packed, the resultant solution were stored at −20° C. for long-term storage, and can be stored in a 2-8° C. freezer for short-term use;

2× HBS Solution:

0.25% (m/V) Trypsin solution: 2.50 g of Trypsin and 0.20 g of EDTA were weighed and placed into a 1000 ml beaker, was added with 900 ml 1× PBS to dissolve, a 1000 ml graduated cylinder was used to dilute to 1000 ml after completing the dissolution, disposable syringe filter (0.22 μm, PES) was used to filter to sterilize, the resultant solution was packed and stored in a 50 ml centrifuge tube, can be stored in a −20° C. refrigerator for long-term storage, and in a 2-8° C. refrigerator for short-term use;

1 M NaOH Solution

1.5 M NaCl Solution, 1 M NaCl Solution, 0.15 M NaCl Solution:

1 M Tris-HCl (pH 6-8) Solution

250 mM Tris-HCl (pH 6-8) Solution:

25 mM Tris-HCl (pH 6-8) Solution:

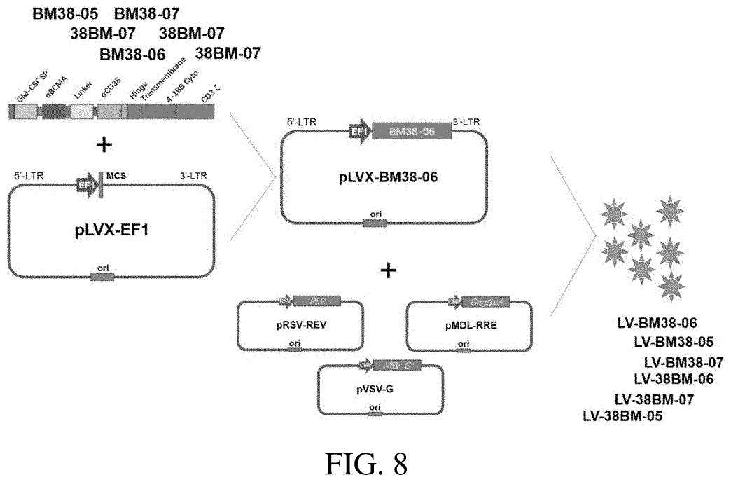

The flow for constructing the recombinant lentivirus according to the present invention was shown in FIG. 8.

1. Construction of Recombinant Lentiviral Backbone Plasmids

As shown in the above figure, the sequences of the fully synthetic chimeric antigen receptors (BM38-05, BM38-06, BM38-07, 38BM-05, 38BM-06, 38BM-07) based on BCMA, CD38 dual targets were constructed to the multiple cloning site MCS at downstream of the promoter EF1 of lentiviral backbone plasmid pLVX-EF1 by double digestion and re-ligation to obtain recombinant lentiviral backbone plasmids pLVX-EF1-BM38-05, pLVX-EF1-BM38-06, pLVX-EF1-BM38-07, pLVX-EF1-38BM-05, pLVX-EF1-38BM-06, pLVX-EF1-38BM-07, the enzyme cleavage sites were 5′-BamHI, 3′-XhoI. The constructed recombinant backbone plasmids were prepared in a large-scale for recombinant lentiviral packaging after verification by sequencing without errors.

2. Preparation in a Large-Scale of Recombinant Lentiviral Backbone Plasmids and Packaging Plasmids

For the constructed lentiviral backbone plasmids and other packaging plasmids above, a standard strain library was established, and then the strains were activated and cultured in large numbers. The thalli were collected, and the above plasmids were extracted and prepared by the method in guideline for EndoFree Maxi Plasmid Kit and plasmid extraction kits for subsequent cell transfection and recombinant virus packaging.

3. Cultivation of LentiX-293T Cells

1) The frozen LentiX-293 T cells were taken out from a liquid nitrogen tank, rapidly transferred to a 37° C. water bath for 1-2 min and then transferred to a biological safety cabinet; 9 ml of pre-cooled complete medium containing 10% FBS was in advance added into a 15 ml centrifuge tube, the cell sap in a cryogenic vial slowly transferred to a 15 ml centrifuge tube with 1 ml pipette, and centrifuged for 10 minutes at 1000 g and the supernatant was discarded, the precipitate was re-suspended with 3 ml of complete medium, and then transferred to a 10 cm petri dish, supplemented with complete medium containing 10% FBS to 8 ml/10 cm dish, after 24 hours, the cells were observed by a microscope, and the cells were passaged again when the cell confluence reached about 70%.

2) LentiX-293 T cells, which are free from contamination and in a good condition, were selected, 5 petri dishes was set as one group, after the cells were trypsinized, 8 ml complete medium was drawn with an electric pipette, 2 ml, was added into each digested petri dish to prevent the petri dish from drying, all cells are blown into a single cell suspension using a 10 ml pipette and transferred into a T75 culture medium flask;

3) The remaining cells in the 5 petri dishes were transferred to a culture flask and transferred to a culture flask after rinsing again with the culture medium;

4) The cap of the culture flask was covered tightly, the cell suspension was mixed well in upside-down or by the electric pipette, a proper amount of cell suspension was taken for counting after dilution, the cell suspension was evenly distributed into 20 of 10 cm petri dishes according to the counting result to ensure that each plate has a cell density of about 4×10⁶ cell/10 ml, a cross-shaped shaking was performed for several times along the front-and-back and the left-and-right direction to make the cells spread out, and the cell suspension was cultured in a 5% (v/v) CO₂ incubator with a saturated humidity.

5) The passaged cells were checked, the culture medium for cells was changed when the cell confluence reached 70-80%, the cells adhere well, the cell contour was full and the cells were evenly distributed at the bottom of the petri dishes. The culture medium was replaced with 8 ml of pre-warmed fresh complete medium.

4. Cell Transfection of LentiX-293T

6) ADNA/PEI solution was prepared in a proportion of 1:1 (v/v), the following operations were implemented in a biosafety cabinet in accordance with a sterile operation standard. The amount of LentiX-293 T cells transfected plasmid in each dish was used according to the following proportions: recombinant lentiviral backbone plasmid (20 μg), pRSV-REV (15 μg), pMDL-RRE (10 μg), pVSV-G (7.5 μg). A new 5 ml centrifuge tube was taken, added with the packaging plasmids in the above amount and supplied with DMEM to 1.0 ml, covered, and mixed fully;

7) The transfection was carried out in accordance with the operation on PEI transfection kits

8) After 72 hours, the supernatant from the same dish was collected again together, the supernatant collected at this time contained recombinant lentivirus LV-BM38-07, LV-BM38-06, LV-BM38-05, LV-38BM-05, LV-38BM-06, LV-38BM-07.

5. Purification of Recombinant Lentiviral by Ion Exchange Chromatography

1) The collected supernatant was filtered by a vacuum pump through a 0.22 μm-0.8 μm PES filter to remove impurities and cellular fractions;

2) The supernatants were added with 1.5 M NaCl 250 mM Tris-HCl (pH 6-8) in a ratio of 1:1-1:10;

3) Two ion exchange columns were placed in series, sequentially passed through a column with 4 ml 1 M NaOH, 4 ml 1 M NaCl, 5 ml 0.15 M NaCl 25 mM Tris-HCl (pH 6-8) solution;

4) The solution obtained in the step 2 was loaded onto an ion exchange column through a peristaltic pump at speed of 1-10 ml/min;

5) After all of supernatant was passed through the column, washed once with 10 ml 0.15 M NaCl 25 mM Tris-HCl (pH 6-8) solution;

6) 1-5 ml 1.5 M NaCl 25 mM Tris-HCl (pH 6-8) was used to elute according to the loading amount, the eluate was collected;

7) The eluate was distributed into 50 μL per tube and frozen in a −80° C. refrigerator for long-term storage.

6. Determination of Recombinant Lentivirus Titer

1) 293 T cells were inoculated in a 24-well plate with 5×10⁴ cell/well, the volume of the added medium was 500 ul, different types of cells had different growth rates, and the cell fusion rate during virus infection was 40% -60%;

2) 3 sterile EP tubes were prepared, each tube was charged with 90 ul of fresh complete medium (high glucose DMEM+10% FBS), 24 hours after inoculation, two wells of cells were taken and counted with a hemocytometer to determine the actual number of cells at the time of infection, the number was referred to as N;

3) 10 ul of the virus stock to be measured was taken and added to the first tube, gently mixed, 10 ul of the solution was taken and added to the second tube, and then the operation was continued until the last tube; 410 ul of complete medium (high Sugar DMEM+10% FBS) was added in each tube, the final volume was 500 ul;

4) 20 hours after the beginning of infection, the supernatant was removed, replaced with 500 μl of complete medium (high sugar DMEM+10% FBS), and cultured continuously for 48 hours with 5% CO₂;

5) After 72 hours, the fluorescence expression was observed, under normal conditions, the number of fluorescent cells decreased with increase of dilution fold, and then photographed;

6) The cells were digested with 0.2 ml 0.25% trypsin-EDTA solution, placed at 37° C. for 1 min. The entire cell surface was purged with the medium, the cells were harvested by centrifugation. Genomic DNA was extracted according to the instruction on DNeasy kit. Each sample tube was charged with 200 μl eluent to wash DNA and quantitated;

7) Preparation of target DNA detection qPCRmix master I (the sequences of qPCR primer were SEQ ID NO:191-SEQ ID NO.196):

EF1 α-F:
(SEQ ID NO: 191)
5-TATCGATGCTCCGGTGCCCGTCAGT-3
EF1 α-R:
(SEQ ID NO: 192)
5-TCACGACACCTGAAATGGAAGA-3
WPRE-qPCR-F:
(SEQ ID NO: 193)
5-TCCGGGACTTTCGCTTT-3
WPRE-qPCR-R:
(SEQ ID NO: 194)
5-CAGAATCCAGGTGGCAACA-3
Actin-qPCR-F:
(SEQ ID NO: 195)
5-CATGTACGTTGCTATCCAGGC-3
Actin-qPCR-R:
(SEQ ID NO: 196)
5-TCCTTAATGTCACGCACGAT-3

The components of the qPCRmix Master I was shown in Table 5. In Table 5, N=number of reactions. For example: the total reaction number was 40, 1 ml 2× TaqMan Universal PCR Master Mix, 4 μl forward primer, 4 μl reverse primer, 4 μl probe and 788 μl H₂O were mixed, shaken and then placed on ice.

Preparation of internal control DNA detection qPCRmix tube II (the sequences of qPCR primer were SEQ ID NO:194, SEQ ID NO.195):

(SEQ ID NO: 194)
Actin-qPCR-F: 5-CATGTACGTTGCTATCCAGGC-3
(SEQ ID NO: 195)
Actin-qPCR-R: 5-TCCTTAATGTCACGCACGAT-3

The components of the qPCRmix Master II was shown in Table 6. In Table 6, N=number of reactions. For example: the total reaction number was 40, 1 ml 2× TaqMan Universal PCR Master Mix, 100 μl 10×RNaseP primer/probe mix and 700 μl H₂O were mixed, shaken and then placed on ice.

The PCR system was established on a pre-cooled 96-well PCR plate. 45 μl was taken from Master I to add into the wells of each row of A-D, 45 μl was taken from Master I to add into the wells of each row of E-G.

5 μl of plasmid standard and genomic DNA of the samples to be tested were added to the rows of A-D, and each sample was repeated once. 1 more well was left to add with 5 μl of water as a no-template control.

5 μl of genomic standard and genomic DNA of the samples to be tested were added to the rows of E-G, and each sample was repeated once. 1 more well was left to add with 5 μl of water as a no-template control.

The used quantitative PCR instrument was Roche LC96 quantitative system. The cycling conditions were set into: 94° C., 3 minutes, followed by 94° C., 15 seconds, 60° C., 1 minute, 40 cycles, finally 72° C., 3 minutes till the procedure terminated.

Data analysis: the measured copy number of the integrated lentiviral vector in the DNA samples was calibrated with the number of genomes to obtain the number of integrated virus copies per genome.

Titers (integration units per ml, IU ml-l) were calculated by the following formula:

IU/ml=(C×N×D×1000)/V

Wherein: C=Average number of integrated virus copies per genome

N=Number of cells at the time of infection (about 1×10⁵)

D=Dilution fold of virus vectors

V=Volume of the added diluted virus

The titer determination result of the recombinant lentiviral LV-BM38-07, LV-BM38-06, LV-BM38-05, LV-38BM-05, LV-38BM-06, LV-38BM-07 containing CAR genes was shown in Table 7.

7. Titer Determination of Recombinant Lentiviral Inflected Jurkat Cells

The virus titers of the recombinant lentivirus solution purified by an ion exchange chromatography were evaluated by transducing Jurkat cells and CAR expression or expression of marker genes.

Jurkat cells were transduced with 3-fold serial dilutions of virus supernatant on the first day with a starting concentration of 1:300. CAR expression was evaluated on the 5^{th} day with BCMA-Fc antigen (AcroBiosystem). The virus titer was calculated according to the following formula:

(% CAR+)×(# Jurkat cells)/(viral load (ml))×(dilution)

The average virus titer was calculated by the dilution points in the positive linear range of 1 to 20% CAR.

The titers of BCMA-Fc recombinant lentivirus-infected Jurkar cells were shown in Table 8.

### EXAMPLE 8

### Isolation and Cultivation of T Cells and Transduction of T Cells by Lentivirus

50 ml of fresh peripheral blood was drawn from healthy volunteers, human peripheral blood mononuclear cells (PBMC) were obtained through a conventional method. The cells were washed once with an appropriate amount of MACS buffer (in 1.5 ml/10⁷ PBMC), centrifuged for 10 min at 800 r/min to precipitate the cells, the supernatant was discarded. The cells were re-suspended (in 80 μl/10⁷ PBMC) with an, appropriate amount of MACS buffer, and then added with an appropriate amount of anti-human-CD3 immuno-magnetic beads (in 20 μl/10⁷ PBMC) and mixed evenly, incubated for 15 min at 4° C. Then the cells were washed with an appropriate amount of MACS buffer (in 1.5 ml/10⁷ PBMC), centrifuged for 10 min at 800 r/min to precipitate the cells, the supernatant was discarded. The cells were re-suspended with 500 μL MACS buffer. A MS separation column was placed on a MiniMACS separator, and the separation column was washed once with a 500 μL MACS buffer. The cell suspension was added onto the MS separation column. The cells that flowed out firstly were CD3-cells not labeled with the magnetic beads. The separation column was washed with 500 μL MACS buffer three times, the MS separation column is removed from the MiniMACS separator, and placed into a 15 ml centrifuge tube. 1 ml MACS buffer was added into the separation column, the retained cells were quickly eluted, and the eluate was the isolated CD3+T lymphocytes. An appropriate amount of MACS buffer was added, mixed well and counted. The supernatant was discarded after centrifugation at 1000 r/min for 10 min. The cells was re-suspended with a RPMI1640 medium containing 10% FBS, the cell concentration was adjusted to 1×10⁶/ml in a 6-well plate. The plate was then placed a 37° C., 5% CO₂ incubator.

T lymphocytes were activated according to the instruction of human T lymphocytes CD3/CD28 immunoactivated magnetic beads. The isolated CD3+T lymphocytes were plated on a 24-well plate at 1×10⁶ cells/well. 25 μL of pre-washed magnetic beads were added to each well, and add with recombinant human IL-2 (purchased from Shanghai Huaxin Biological High Technology Co., Ltd.) till the final concentration was 30 U/ml. The 24-well plate was cultured in a 37° C., 5% CO₂ incubator. The medium containing recombinant human IL-2 was changed every 2-3 days. The passage was performed depending on the cell growth density.

Recombinant lentivirus infection can be performed when CD3+T lymphocytes grew in a good condition at a density of about 2×10⁶ cells/ml. The infected MOI was calculated based on the titer of recombinant lentivirus infected Jurkat cells, which generally did not exceed 5. Polybrene was added to a 24-well plate till the final concentration was 4 μg/ml, at the same time the lentiviral suspensions LV-BM38-07, LV-BM38-06, LV-BM38-05, LV-38BM-05, LV-38BM-06, LV-38BM-07 were added, after 6 hours, the fresh culture medium was supplemented or the medium was changed completely to continue culturing.

### EXAMPLE 9

### Detection of BM38-05, BM38-06, BM38-07 CAR-T Cells

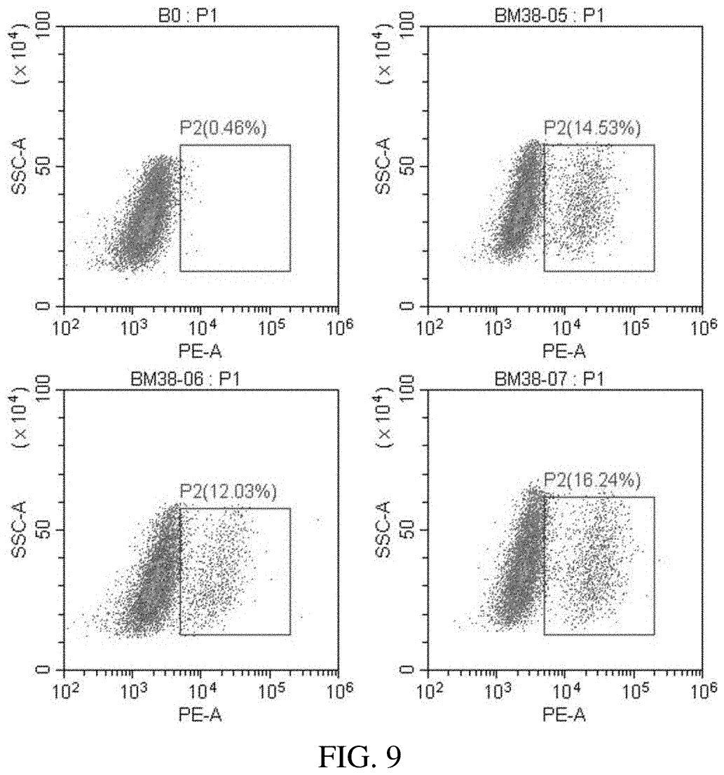

The expression of chimeric antigen receptors was detected by flow cytometry on the 6^{th} day of the cultivation of infected T cells. Firstly, the infected CAR-T cells were incubated with biotin-labeled human Fc-BCMA protein for 30 min at 37° C., washed twice with D-PBS, and then added with PE-labeled Streptavidin and incubated at 37° C. for 30 min. After washing with D-PBS 3 times, the ratio of positive cells was detected by flow cytometry. Non-infected T lymphocytes and T-cells only stained with PE-labeled Streptavidin were used as negative controls to identify T-cells infected with the chimeric antigen receptors and their positive rate. The results were shown in FIG. 9.

In addition, the marker proteins or tags such as GFP can be expressed simultaneously during constructing a CAR expression vector, the positive rate of T cells expressing the marker proteins such as GFP was confirmed by fluorescence imaging or FACS analysis. By referring to the positive level of Fc-BCMA recombinant antigen for T cells expressing CAR genes, it was confirmed that the lentiviral-infected T cells carrying CAR genes can successfully display the CAR structure on the cell membrane of the T cells. Furthermore, there was no significant difference in the positive rate between the two detection methods, which indicated that T cells stably expressing chimeric antigen receptors can be obtained by lentiviral transduction.

### EXAMPLE 10

### In Vitro Toxicity Study of T Lymphocytes Expressing Chimeric Antigen Receptors

Target cells with tumor-specific antigens and T lymphocytes or CAR-T cells were mixed with into a suitable in vitro cell culture system. After a period of time, T cells or CAR-T cells can be observed to recognize (aggregate) and kill (reduce the number of tumor cells) the target cells. For example, in this experiment, BCMA-overexpressing K562 cells were mixed with T cells or BM38-05, BM38-06, BM38-07 CAR-T. The cells used in this experiment were K562 (K562 cells themselves did not express BCMA proteins) and K562 cells (K562-BCMA) transfected with BCMA as target cells. The effector cells were CAR-T cells prepared as described above. The effector-to-target ratio was 2.5, 12.5:1 and 25:1, and the number of the target cells was 10,000 cells/well. Appropriate number of effector cells can be added depending on different effector-to-target ratio. All cells were cultured in a cell culture incubator at 5% CO₂ and 37° C. At the beginning of the experiment, K562-BCMA cells at 10,000 cell/well firstly were inoculated into a 6-well plate, and then an appropriate number of T cells or CAR-T cells was added depending on the effector-to-target ratio after 12 h of incubation. The incubation was continued in a 5% CO₂, 37° C. incubator for 4 h, the culture plate was taken out and observed under an inverted microscope to calculate the killing efficiency. The results were shown in Table 9. BM38-05, BM38-06, and BM38-07 had a significant killing effect on BCMA-positive K562 cells. In contrast, 38BM-05, 38BM-06, 38BM-07 had a significantly consistent and weak killing effect.

### EXAMPLE 11

### In Vitro Toxicity Study of T Lymphocytes Expressing Chimeric Antigen Receptors

When CAR-T cells kill tumor cells, T cells firstly recognize and identify the tumor cells carrying tumor antigens, form immune synapses, and then release killing factors to finish the immune monitoring and killing of the T cells, therefore, the process was a continuous process. Traditional method for detecting the killing of T cells on tumor cells is to perform endpoint detection by taking one time point, however, the results obtained by such method often have larger errors and also cannot truly reflect the complete process for killing tumor cells as for T cells. In order to overcome the technical bottleneck and restriction of such detection method, a real time cellular analysis (RTCA) technology can carry out a real-time, label-free, dynamic monitoring on cytotoxic effects caused by small molecule compounds, antibody drugs, T cells, etc based on the detection of electrical impedance generated by cells attached to the culture plate with microelectrodes. Based on such real-time cell killing analysis technology of electrical impedance, researchers can obtain high-sensitivity quantitative data, which will be helpful to study and reveal the specific mechanism of antitumor compounds or cells, and also can significantly reduce the cost of the experiments and increase the accuracy of the results.

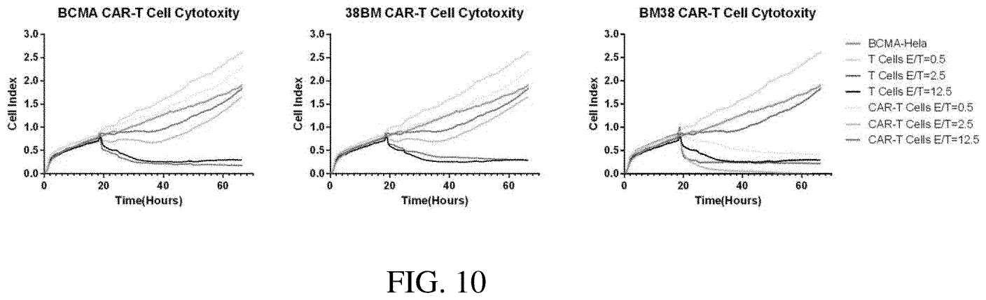

The cells in this experiment were human cervical cancer cell lines Hela and BCMA-transfected Hela cells (BCMA-Hela) as target cells, and the effector cells were the BM38-06CAR-T cells by prepared previously. The effector-to-target ratio was 2.5, 12.5:1, and 25:1 respectively, the number of target cells was 10,000 cells/well, and an appropriate number of effector cells were added according to different effector-to-target ratios. All cells were cultured in a 5% CO₂, 37° C. cell incubator. RTCA technology was used to monitor continuously and dynamically the adherence, spreading, and reproduction of target cells. Cell Index was used to represent the growth status of target cells, or can be equivalent to the number of target cells. Throughout the whole experimental process with the exception of the addition process of effector cells, it shall be ensured that the culture plates and detection equipment were in a stable culture environment to avoid large fluctuations in the Cell Index data. In order to obtain a time-dependent cell effect characteristic curve, 90 ul of medium was added to wells in a 16-well E-plate culture. After the background baseline was detected, 100 ul of cell suspension was added, mixed well and placed onto the test bench in a CO₂ incubator to obtain continuously the Cell Index of the cell growth status. After 18 hours, target cells were allowed to grow adherently for a period of time, and then added with an equal volume of effector cell suspension, and placed onto the test bench in an incubator to continue to collect the Cell Index data that reflects the cell growth status. After 48 hours, the whole experiment was finished, and the collected data was exported and plotted. The results were shown in FIG. 10.

### EXAMPLE 12

### In Vivo Antitumor Effect of CAR-T Cells (Survival Curve of an Experimental Animal Model)

In order to confirm if CAR-T cells, which have a good, killing effect to tumor cells in vitro, still have killing effect to tumor cells in a tumor-bearing mouse model. The example established a mouse model by injecting with K562-BCMA cells and control K562 cells through the tail vein, the model mouse were injected with different numbers of BCMA CAR-T cells, BM38 CAR-T cells and control T cells again through the tail vein one week later. The survival time of the model mouse was observed, and the mouse was killed at a certain time point, the peripheral blood and bone marrow tissue samples of the mouse were collected, the number of CAR-T cells and tumor cells were measured by flow cytometry to evaluate the tumor killing ability of different CAR-T Cells in the tumor-bearing mouse model.

The experimental process was as follows: K562-BCMA cells were grown in a RPMI1640 medium containing 10% heat-inactivated fetal bovine serum as described above. B-NDG® mouse (NOD-PrkdcscidIL2rgtm1/Bcgen) was purchased from Beijing Biocytogen Co., Ltd., bred for 1 week under sterile conditions and were injected with 5×10⁵ cells/100 μl of K562-BCMA cells through the tail vein to establish a tumor-bearing mouse model.

The mouse was reinfused with 5×10⁶ control T cells or CAR-T cells at 7-8 days after implantation of the tumor cells. The T cells or CAR-T cells were partially thawed in a 37° C. water bath and then completely thawed by adding 1 ml of cold sterile PBS to the tube containing the cells. The thawed cells were transferred to a 15 ml falcon tube and adjusted to a final volume of 10 ml with PBS. The cells were washed twice at 1000 rpm for 10 minutes each and then counted by a hemocytometer. CAR T cells were normalized relative to CAR transduction such that each group had the same percentage of CAR+T cells. 5×10⁶ cells was re-suspended with cold PBS at a concentration of 50×10⁶ cells/ml and kept on ice until the mouse were administered. The mouse was injected intravenously with 100 μl of CAR T cells through the tail vein at a dose of 5×10⁶ T cells per mouse.

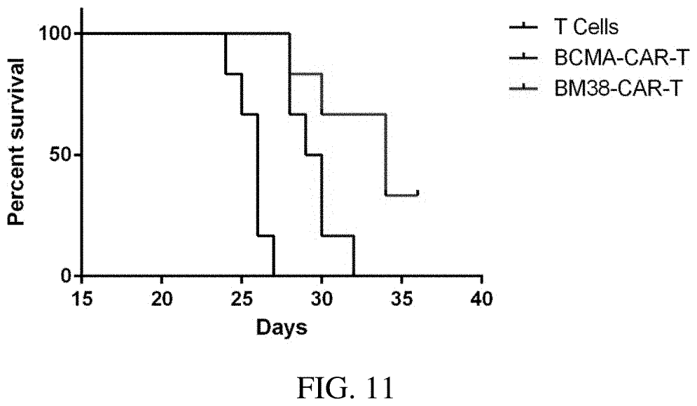

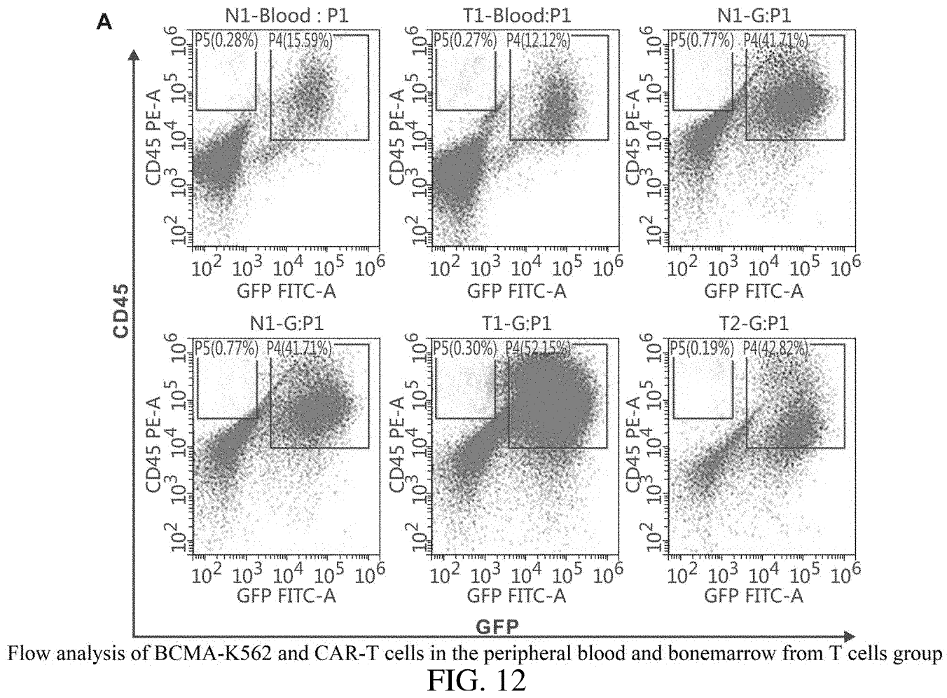

5 to 7 mice in each group were treated with 100 μl PBS alone (PBS), untransduced T cells (Mock), BCMA CAR-T cells or BM38 CAR-T cells, T cells were provided by the same healthy volunteers. After the injection of CAR-T, the survival status of the experimental animals was measured daily, and the weight changes were recorded. The mouse was judged as appearance of a death event if extremely poor survival status such as inability to eat, paralysis and other symptoms was monitored, at this time, the peripheral blood and bone marrow samples were collected after the mouse was sacrificed, and the number of tumor cells (K562-BCMA, GFP positive) and CAR-T cells (human CD45+CAR+) in the peripheral blood and bone marrow was analyzed by flow cytometry. The time of the appearance of the death event was recorded, and the survival time curve of mice in each group was statistically analyzed. The results were shown in FIG. 11. The results show that the tumor-bearing mice reinfused with BM38 CAR-T cells have a longer lifetime.

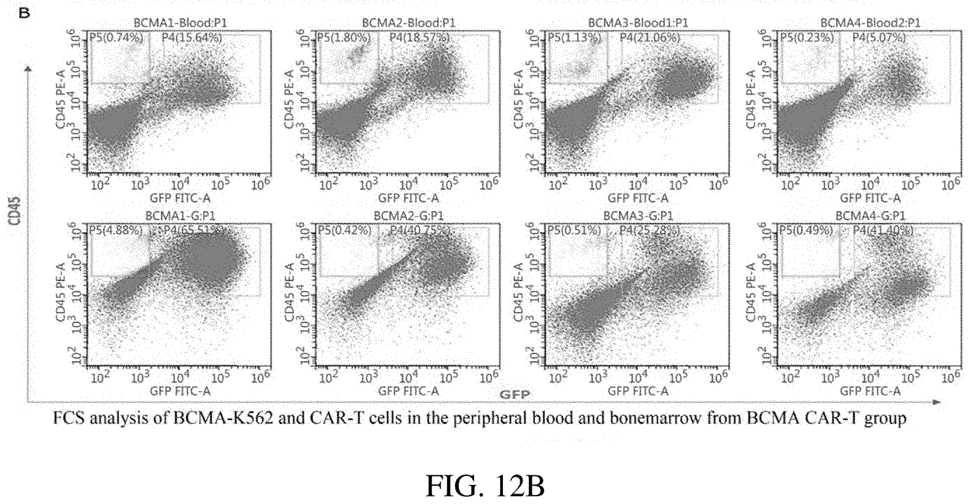

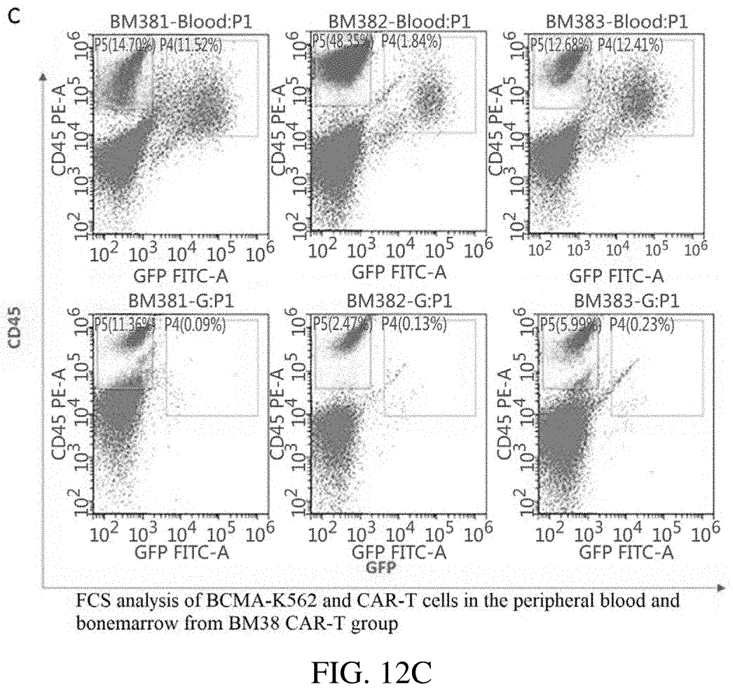

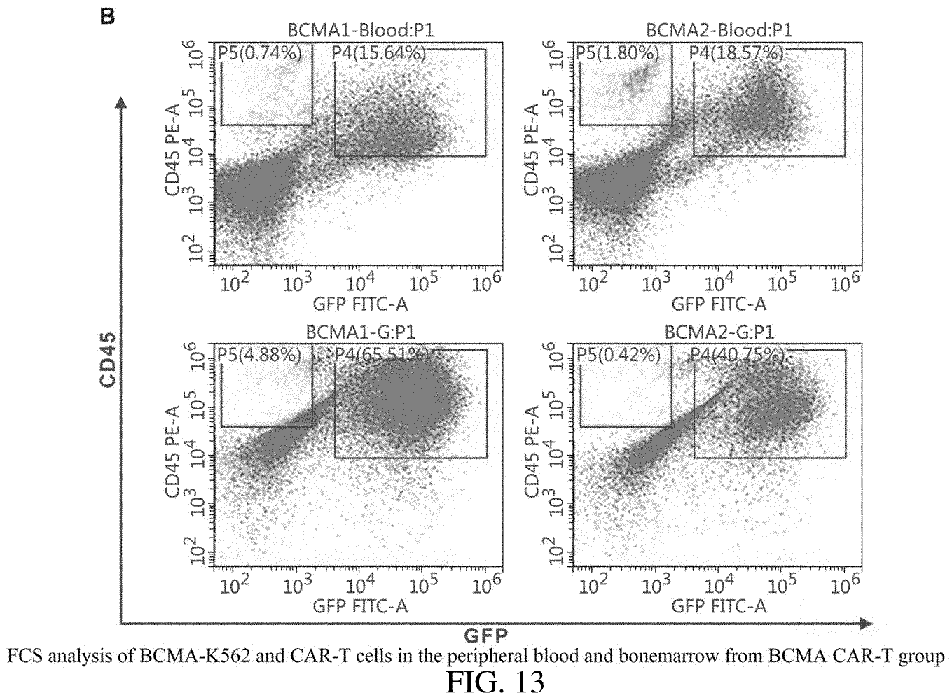

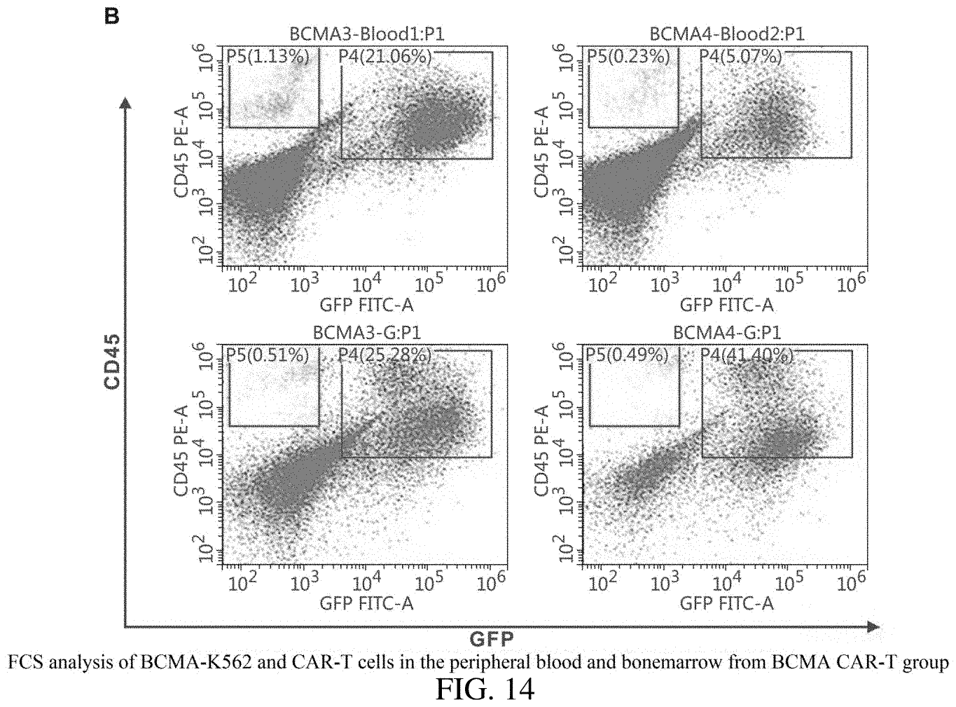

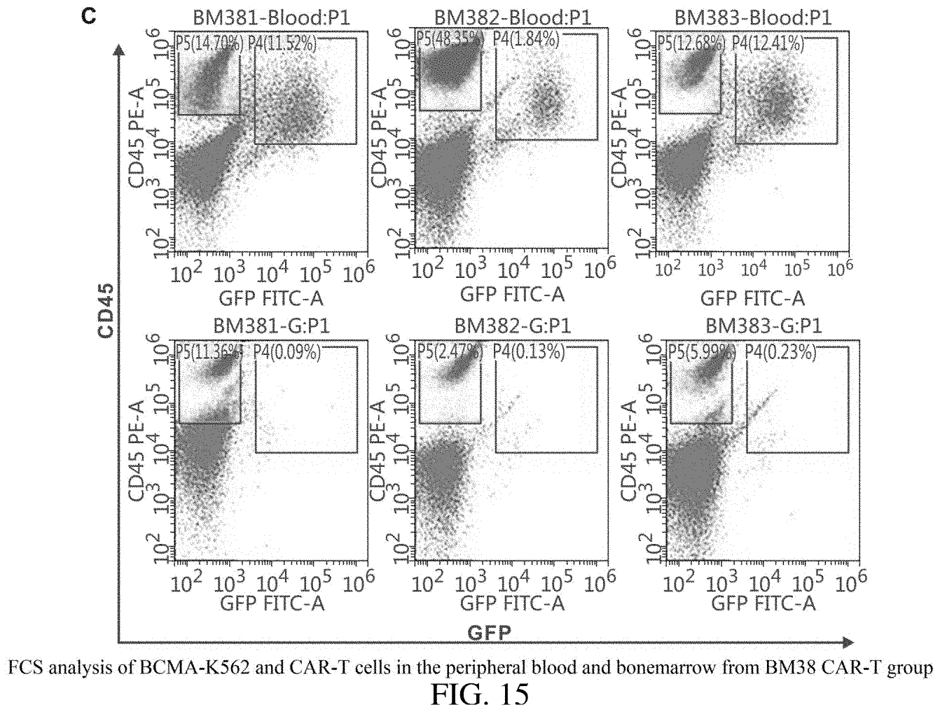

Flow cytometry analysis of the peripheral blood and bone marrow in the tumor-bearing mice showed that the peripheral blood and bone marrow in the tumor-bearing mice reinfused with BM38 CAR-T cells had fewer K562-BCMA cells but at the same time had more CAR-T cells, as shown in FIGS. 12-15.

While the preferred embodiments of the present invention have been described, those skilled in the art can make various changes and modifications to these embodiments upon knowing the basic inventive concepts. Therefore, the appended claims are intended to be construed as embodying preferred embodiments and all modifications and alternative constructions which fall within the scope of the invention.

Obviously, those skilled in the art can make various modifications and variations to the present invention without departing from the spirit and scope of the present invention. Thus, it is intended that the present invention covers such modifications and variations as come within the scope of the appended claims and their equivalents.

The appended tables in the present invention are as follows:

TABLE 1
Amino Acid Codons

| Chinese | | Three-letter | Single-letter | |
|---|---|---|---|---|
| Name | English Name | Abbreviation | Abbreviation | Nucleotide Codon |
| [symbol] | Glycine | Gly | G | GGU, GGC, GGA, GGG |
| [symbol] | Alanine | Ala | A | GCU, GCC, GCA, GCG |
| [symbol] | Valine | Val | V | GUU, GUC, GUA, GUG |
| [symbol] | Leucine | Leu | L | CUU, CUC, CUA, CUG, UUA, UUG |
| [symbol] | Isoleucine | Ile | I | AUU, AUC, AUA |
| [symbol] | Proline | Pro | P | CCU, CCA, CCG, CCC |
| [symbol] | Phenylalanine | Phe | F | UUU, UUC |
| [symbol] | Tyrosine | Tyr | Y | UAJ, UAC |
| [symbol] | Tryptophan | Trp | W | UGG |
| [symbol] | Serine | Ser | s | UCU, UCA, UCC, UCG, AGU, AGC |
| [symbol] | Threonine | Thr | T | ACU, ACC, ACG, ACA |
| [symbol] | Cystine | Cys | C | UGU, UGC |
| [symbol] | Methionine | Met | M | AUG |
| [symbol] | Asparagine | Asn | N | AAU, AAC |
| [symbol] | Glutamine | Gln | Q | CAA, CAG |
| [symbol] | Asparticacid | Asp | D | GAU, GAC |
| [symbol] | Glutamicacid | Glu | E | GAA, GAG |
| [symbol] | Lysine | Lys | K | AAA, AAG |
| [symbol] | Arginine | Arg | R | CGU, CGC, CGG, CGA, AGA, AGG |
| [symbol] | Histidine | His | H | CAU, CAC |
| [symbol] | — | — | — | UAA, UAG, UGA |

TABLE 2
Affinity of recombinant BCMA monoclonal antibody
to Fc-BCMA

| Antibody | Kₐ (1/Ms) | K_{d} (1/s) | K_{D} (M) |
|---|---|---|---|
| BM-3E2 | 3.5 E+05 | 3.6 E−05 | 6.9 E−09 |
| BM-6G3 | 4.8 E+06 | 3.6 E−04 | 5.6 E−09 |
| BM-7A9 | 5.2 E+05 | 1.6 E−02 | 7.3 E−09 |
| BM-4G12 | 6.2 E+04 | 1.5 E−05 | 4.2 E−09 |
| BM-6E7 | 7.3 E+04 | 4.4 E−04 | 9.1 E−10 |
| BM-9B5 | 6.2 E+06 | 5.8 E−04 | 6.5 E−09 |
| BM-4C11 | 3.8 E+05 | 3.2 E−05 | 6.9 E−08 |
| BM-3G7 | 2.9 E+05 | 6.1 E−06 | 7.2 E−09 |
| BM-6F5 | 5.4 E+04 | 7.6 E−05 | 5.4 E−08 |
| BM-5C8 | 2.9 E+05 | 2.9 E−06 | 8.2 E−09 |

TABLE 3
Affinity of recombinant CD38 monoclonal antibody
to rCD38

| Antibody | Ka (1/Ms) | Kd (1/s) | KD (M) |
|---|---|---|---|
| 38-4A6 | 7.2 E+06 | 4.4 E−04 | 2.5 E−06 |
| 38-4B7 | 6.3 E+06 | 5.6 E−05 | 4.2 E−05 |
| 38-5A4 | 8.5 E+05 | 8.2 E−03 | 3.6 E−06 |
| 38-6C9 | 1.9 E+06 | 9.1 E−06 | 5.2 E−07 |
| 38-6D12 | 2.4 E+06 | 6.3 E−05 | 5.1 E−06 |
| 38-3F10 | 5.3 E+04 | 5.7 E−05 | 7.1 E−06 |
| 38-4F9 | 6.7 E+06 | 5.9 E−05 | 5.6 E−05 |
| 38-6B10 | 6.5 E+05 | 6.2 E−06 | 8.6 E−06 |
| 38-7A12 | 5.9 E+04 | 6.8 E−04 | 9.8 E−06 |
| 38-4B5 | 4.8 E+06 | 4.7 E−05 | 7.2 E−05 |

TABLE 4
Design of different CAR constructs

| | | | | | | | Intracellular | | |
|---|---|---|---|---|---|---|---|---|---|
| CAR | SP | Target 1 | Linker | Target 2 | Hinge | TM | Cos1 | Cos2 | Prism |
| BM-01 | GM-CSF | BM-3E2 | | | CD8 | CD8 | CD28 | 4-1BB | CD3ζ |
| BM-02 | GM-CSF | BM-6G3 | | | CD8 | CD8 | CD28 | 4-1BB | CD3ζ |
| BM-03 | GM-CSF | BM-7A9 | | | CD8 | CD8 | CD28 | 4-1BB | CD3ζ |
| BM-04 | GM-CSF | BM-4G12 | | | CD8 | CD8 | CD28 | 4-1BB | CD3ζ |
| BM-05 | GM-CSF | BM-6E7 | | | CD8 | CD8 | CD28 | 4-1BB | CD3ζ |
| BM-06 | GM-CSF | BM-9B5 | | | CD8 | CD8 | CD28 | 4-1BB | CD3ζ |
| BM-07 | GM-CSF | BM-4C11 | | | CD8 | CD8 | CD28 | 4-1BB | CD3ζ |
| BM-08 | GM-CSF | BM-3G7 | | | CD8 | CD8 | CD28 | 4-1BB | CD3ζ |
| BM-09 | GM-CSF | BM-6F5 | | | CD8 | CD8 | CD28 | 4-1BB | CD3ζ |
| BM-10 | GM-CSF | BM-5C8 | | | CD8 | CD8 | CD28 | 4-1BB | CD3ζ |
| 38-01 | GM-CSF | | | 38-4A6 | CD8 | CD8 | CD28 | 4-1BB | CD3ζ |
| 38-02 | GM-CSF | | | 38-4B7 | CD8 | CD8 | CD28 | 4-1BB | CD3ζ |
| 38-03 | GM-CSF | | | 38-5A4 | CD8 | CD8 | CD28 | 4-1BB | CD3ζ |
| 38-04 | GM-CSF | | | 38-6C9 | CD8 | CD8 | CD28 | 4-1BB | CD3ζ |
| 38-05 | GM-CSF | | | 38-6D12 | CD8 | CD8 | CD28 | 4-1BB | CD3ζ |
| 38-06 | GM-CSF | | | 38-3F10 | CD8 | CD8 | CD28 | 4-1BB | CD3ζ |
| 38-07 | GM-CSF | | | 38-4F9 | CD8 | CD8 | CD28 | 4-1BB | CD3ζ |
| 38-08 | GM-CSF | | | 38-6B10 | CD8 | CD8 | CD28 | 4-1BB | CD3ζ |
| 38-09 | GM-CSF | | | 38-7A12 | CD8 | CD8 | CD28 | 4-1BB | CD3ζ |
| 38-10 | GM-CSF | | | 38-4B5 | CD8 | CD8 | CD28 | 4-1BB | CD3ζ |
| BM38-01 | GM-CSF | BM-3E2 | EAAAK | 38-4A6 | CD8 | CD8 | | 4-1BB | CD3ζ |
| BM38-02 | GM-CSF | BM-6G3 | EAAAK | 38-4B7 | CD8 | CD8 | | 4-1BB | CD3ζ |
| BM38-03 | GM-CSF | BM-7A9 | EAAAK | 38-5A4 | CD8 | CD8 | | 4-1BB | CD3ζ |
| BM38-04 | GM-CSF | BM-4G12 | EAAAK | 38-6C9 | CD8 | CD8 | | 4-1BB | CD3ζ |
| BM38-05 | GM-CSF | BM-6E7 | EAAAK | 38-6D12 | CD8 | CD8 | | 4-1BB | CD3ζ |
| BM38-06 | GM-CSF | BM-9B5 | EAAAK | 38-3F10 | CD8 | CD8 | | 4-1BB | CD3ζ |
| BM38-07 | GM-CSF | BM-4C11 | EAAAK | 38-4F9 | CD8 | CD8 | | 4-1BB | CD3ζ |
| BM38-08 | GM-CSF | BM-3G7 | EAAAK | 38-6B10 | CD8 | CD8 | | 4-1BB | CD3ζ |
| BM38-09 | GM-CSF | BM-6F5 | EAAAK | 38-7A12 | CD8 | CD8 | | 4-1BB | CD3ζ |
| BM38-10 | GM-CSF | BM-5C8 | EAAAK | 38-4B5 | CD8 | CD8 | | 4-1BB | CD3ζ |
| BM38-11 | GM-CSF | BM-3E2 | EAAAK | 38-4B7 | CD8 | CD8 | | 4-1BB | CD3ζ |
| BM38-12 | GM-CSF | BM-6G3 | EAAAK | 38-4B7 | CD8 | CD8 | | 4-1BB | CD3ζ |
| BM38-13 | GM-CSF | BM-7A9 | EAAAK | 38-4B7 | CD8 | CD8 | | 4-1BB | CD3ζ |
| BM38-14 | GM-CSF | BM-4G12 | EAAAK | 38-4B7 | CD8 | CD8 | | 4-1BB | CD3ζ |
| BM38-15 | GM-CSF | BM-6E7 | EAAAK | 38-4B7 | CD8 | CD8 | | 4-1BB | CD3ζ |
| BM38-16 | GM-CSF | BM-9B5 | EAAAK | 38-4B7 | CD8 | CD8 | | 4-1BB | CD3ζ |
| BM38-17 | GM-CSF | BM-4C11 | EAAAK | 38-4B7 | CD8 | CD8 | | 4-1BB | CD3ζ |
| BM38-18 | GM-CSF | BM-3G7 | EAAAK | 38-4B7 | CD8 | CD8 | | 4-1BB | CD3ζ |
| BM38-19 | GM-CSF | BM-6F5 | EAAAK | 38-4B7 | CD8 | CD8 | | 4-1BB | CD3ζ |
| BM38-20 | GM-CSF | BM-5C8 | EAAAK | 38-4B7 | CD8 | CD8 | | 4-1BB | CD3ζ |
| BM38-21 | GM-CSF | BM-3E2 | EAAAK | 38-5A4 | CD8 | CD8 | | 4-1BB | CD3ζ |
| BM38-22 | GM-CSF | BM-6G3 | EAAAK | 38-5A4 | CD8 | CD8 | | 4-1BB | CD3ζ |
| BM38-23 | GM-CSF | BM-7A9 | EAAAK | 38-5A4 | CD8 | CD8 | | 4-1BB | CD3ζ |
| BM38 24 | GM-CSF | BM-4G12 | EAAAK | 38-5A4 | CD8 | CD8 | | 4-1BB | CD3ζ |
| BM38-25 | GM-CSF | BM-6E7 | EAAAK | 38-5A4 | CD8 | CD8 | | 4-1BB | CD3ζ |
| BM38-26 | GM-CSF | BM-9B5 | EAAAK | 38-5A4 | CD8 | CD8 | | 4-1BB | CD3ζ |
| BM38-27 | GM-CSF | BM-4C11 | EAAAK | 38-5A4 | CD8 | CD8 | | 4-1BB | CD3ζ |
| BM38-28 | GM-CSF | BM-3G7 | EAAAK | 38-5A4 | CD8 | CD8 | | 4-1BB | CD3ζ |
| BM38-29 | GM-CSF | BM-6F5 | EAAAK | 38-5A4 | CD8 | CD8 | | 4-1BB | CD3ζ |
| BM3S-30 | GM-CSF | BM-5C8 | EAAAK | 38-5A4 | CD8 | CD8 | | 4-1BB | CD3ζ |
| BM38-31 | GM-CSF | BM-3E2 | EAAAK | 38-6C9 | CD8 | CD8 | | 4-1BB | CD3ζ |
| BM38-32 | GM-CSF | BM-6G3 | EAAAK | 38-6C9 | CD8 | CD8 | | 4-1BB | CD3ζ |
| BM38-33 | GM-CSF | BM-7A9 | EAAAK | 38-6C9 | CD8 | CD8 | | 4-1BB | CD3ζ |
| BM38-34 | GM-CSF | BM-4G12 | EAAAK | 38-6C9 | CD8 | CD8 | | 4-1BB | CD3ζ |
| BM38-35 | GM-CSF | BM-6E7 | EAAAK | 38-6C9 | CD8 | CD8 | | 4-1BB | CD3ζ |
| BM38-36 | GM-CSF | BM-9B5 | EAAAK | 38-6C9 | CD8 | CD8 | | 4-1BB | CD3ζ |
| BM38-37 | GM-CSF | BM-4C11 | EAAAK | 38-6C9 | CD8 | CD8 | | 4-1BB | CD3ζ |
| BM38-38 | GM-CSF | BM-3G7 | EAAAK | 38-6C9 | CD8 | CD8 | | 4-1BB | CD3ζ |
| BM38-39 | GM-CSF | BM-6F5 | EAAAK | 38-6C9 | CD8 | CD8 | | 4-1BB | CD3ζ |
| BM38-40 | GM-CSF | BM-5C8 | EAAAK | 38-6C9 | CD8 | CD8 | | 4-1BB | CD3ζ |
| BM38-41 | GM-CSF | BM-3E2 | EAAAK | 38-6D12 | CD8 | CD8 | | 4-1BB | CD3ζ |
| BM38-42 | GM-CSF | BM-6G3 | EAAAK | 38-6D12 | CD8 | CD8 | | 4-1BB | CD3ζ |
| BM38-43 | GM-CSF | BM-7A9 | EAAAK | 38-6D12 | CD8 | CD8 | | 4-1BB | CD3ζ |
| BM38-44 | GM-CSF | BM-4G12 | EAAAK | 38-6D12 | CD8 | CD8 | | 4-1BB | CD3ζ |
| BM38-45 | GM-CSF | BM-6E7 | EAAAK | 38-6D12 | CD8 | CD8 | | 4-1BB | CD3ζ |
| BM38-46 | GM-CSF | BM-9B5 | EAAAK | 38-6D12 | CD8 | CD8 | | 4-1BB | CD3ζ |
| BM38-47 | GM-CSF | BM-4C11 | EAAAK | 38-6D12 | CD8 | CD8 | | 4-1BB | CD3ζ |
| BM38-48 | GM-CSF | BM-3G7 | EAAAK | 38-6D12 | CD8 | CD8 | | 4-1BB | CD3ζ |
| BM38-49 | GM-CSF | BM-6F5 | EAAAK | 38-6D12 | CD8 | CD8 | | 4-1BB | CD3ζ |
| BM38-50 | GM-CSF | BM-5C8 | EAAAK | 38-6D12 | CD8 | CD8 | | 4-1BB | CD3ζ |
| BM38-51 | GM-CSF | BM-3E2 | EAAAK | 38-3F10 | CD8 | CD8 | | 4-1BB | CD3ζ |
| BM38-52 | GM-CSF | BM-6G3 | EAAAK | 38-3F10 | CD8 | CD8 | | 4-1BB | CD3ζ |
| BM38-53 | GM-CSF | BM-7A9 | EAAAK | 38-3F10 | CD8 | CD8 | | 4-1BB | CD3ζ |
| BM38-54 | GM-CSF | BM-4G12 | EAAAK | 38-3F10 | CD8 | CD8 | | 4-1BB | CD3ζ |
| BM38-55 | GM-CSF | BM-6E7 | EAAAK | 38-3F10 | CD8 | CD8 | | 4-1BB | CD3ζ |
| BM38-56 | GM-CSF | BM-9B5 | EAAAK | 38-3F10 | CD8 | CD8 | | 4-1BB | CD3ζ |
| BM38-57 | GM-CSF | BM-4C11 | EAAAK | 38-3F10 | CD8 | CD8 | | 4-1BB | CD3ζ |
| BM38-58 | GM-CSF | BM-3G7 | EAAAK | 38-3F10 | CD8 | CD8 | | 4-1BB | CD3ζ |
| BM38-59 | GM-CSF | BM-6F5 | EAAAK | 38-3F10 | CD8 | CD8 | | 4-1BB | CD3ζ |
| BM38-60 | GM-CSF | BM-5C8 | EAAAK | 38-3F10 | CD8 | CD8 | | 4-1BB | CD3ζ |
| BM38-61 | GM-CSF | BM-3E2 | EAAAK | 38-4F9 | CD8 | CD8 | | 4-1BB | CD3ζ |
| BM38-62 | GM-CSF | BM-6G3 | EAAAK | 38-4F9 | CD8 | CD8 | | 4-1BB | CD3ζ |
| BM38-63 | GM-CSF | BM-7A9 | EAAAK | 38-4F9 | CD8 | CD8 | | 4-1BB | CD3ζ |
| BM38-64 | GM-CSF | BM-4G12 | EAAAK | 38-4F9 | CD8 | CD8 | | 4-1BB | CD3ζ |
| BM38-65 | GM-CSF | BM-6E7 | EAAAK | 38-4F9 | CD8 | CD8 | | 4-1BB | CD3ζ |
| BM38-66 | GM-CSF | BM-9B5 | EAAAK | 38-4F9 | CD8 | CD8 | | 4-1BB | CD3ζ |
| BM38-67 | GM-CSF | BM-4C11 | EAAAK | 38-4F9 | CD8 | CD8 | | 4-1BB | CD3ζ |
| BM38-68 | GM-CSF | BM-3G7 | EAAAK | 38-4F9 | CD8 | CD8 | | 4-1BB | CD3ζ |
| BM38-69 | GM-CSF | BM-6F5 | EAAAK | 38-4F9 | CD8 | CD8 | | 4-1BB | CD3ζ |
| BM38-70 | GM-CSF | BM-5C8 | EAAAK | 38-4F9 | CD8 | CD8 | | 4-1BB | CD3ζ |
| BM38-71 | GM-CSF | BM-3E2 | EAAAK | 38-6B10 | CD8 | CD8 | | 4-1BB | CD3ζ |
| BM38-72 | GM-CSF | BM-6G3 | EAAAK | 38-6B10 | CD8 | CD8 | | 4-1BB | CD3ζ |
| BM38-73 | GM-CSF | BM-7A9 | EAAAK | 38-6B10 | CD8 | CD8 | | 4-1BB | CD3ζ |
| BM38-74 | GM-CSF | BM-4G12 | EAAAK | 38-6B10 | CD8 | CD8 | | 4-1BB | CD3ζ |
| BM38-75 | GM-CSF | BM-6E7 | EAAAK | 38-6B10 | CD8 | CD8 | | 4-1BB | CD3ζ |
| BM38-76 | GM-CSF | BM-9B5 | EAAAK | 38-6B10 | CD8 | CD8 | | 4-1BB | CD3ζ |
| BM38-77 | GM-CSF | BM-4C11 | EAAAK | 38-6B10 | CD8 | CD8 | | 4-1BB | CD3ζ |
| BM38-78 | GM-CSF | BM-3G7 | EAAAK | 38-6B10 | CD8 | CD8 | | 4-1BB | CD3ζ |
| BM38-79 | GM-CSF | BM-6F5 | EAAAK | 38-6B10 | CD8 | CD8 | | 4-1BB | CD3ζ |
| BM38-80 | GM-CSF | BM-5C8 | EAAAK | 38-6B10 | CD8 | CD8 | | 4-1BB | CD3ζ |
| BM38-81 | GM-CSF | BM-3E2 | EAAAK | 38-7A12 | CD8 | CD8 | | 4-1BB | CD3ζ |
| BM38-82 | GM-CSF | BM-6G3 | EAAAK | 38-7A12 | CD8 | CD8 | | 4-1BB | CD3ζ |
| BM38-83 | GM-CSF | BM-7A9 | EAAAK | 38-7A12 | CD8 | CD8 | | 4-1BB | CD3ζ |
| BM38-84 | GM-CSF | BM-4G12 | EAAAK | 38-7A12 | CD8 | CD8 | | 4-1BB | CD3ζ |
| BM38-85 | GM-CSF | BM-6E7 | EAAAK | 38-7A12 | CD8 | CD8 | | 4-1BB | CD3ζ |
| BM38-86 | GM-CSF | BM-9B5 | EAAAK | 38-7A12 | CD8 | CD8 | | 4-1BB | CD3ζ |
| BM38-87 | GM-CSF | BM-4C11 | EAAAK | 38-7A12 | CD8 | CD8 | | 4-1BB | CD3ζ |
| BM38-88 | GM-CSF | BM-3G7 | EAAAK | 38-7A12 | CD8 | CD8 | | 4-1BB | CD3ζ |
| BM38-89 | GM-CSF | BM-6F5 | EAAAK | 38-7A12 | CD8 | CD8 | | 4-1BB | CD3ζ |
| BM38-90 | GM-CSF | BM-5C8 | EAAAK | 38-7A12 | CD8 | CD8 | | 4-1BB | CD3ζ |
| BM38-91 | GM-CSF | BM-3E2 | EAAAK | 38-4B5 | CD8 | CD8 | | 4-1BB | CD3ζ |
| BM38-92 | GM-CSF | BM-6G3 | EAAAK | 38-4B5 | CD8 | CD8 | | 4-1BB | CD3ζ |
| BM38-93 | GM-CSF | BM-7A9 | EAAAK | 38-4B5 | CD8 | CD8 | | 4-1BB | CD3ζ |
| BM38-94 | GM-CSF | BM-4G12 | EAAAK | 38-4B5 | CD8 | CD8 | | 4-1BB | CD3ζ |
| BM38-95 | GM-CSF | BM-6E7 | EAAAK | 38-4B5 | CD8 | CD8 | | 4-1BB | CD3ζ |
| BM38-96 | GM-CSF | BM-9B5 | EAAAK | 38-4B5 | CD8 | CD8 | | 4-1BB | CD3ζ |
| BM38-97 | GM-CSF | BM-4C11 | EAAAK | 38-4B5 | CD8 | CD8 | | 4-1BB | CD3ζ |
| BM38-98 | GM-CSF | BM-3G7 | EAAAK | 38-4B5 | CD8 | CD8 | | 4-1BB | CD3ζ |
| BM38-99 | GM-CSF | BM-6F5 | EAAAK | 38-4B5 | CD8 | CD8 | | 4-1BB | CD3ζ |
| BM38-100 | GM-CSF | BM-5C8 | EAAAK | 38-4B5 | CD8 | CD8 | | 4-1BB | CD3ζ |

TABLE 5
Components in qPCRmix Master I

| | | |
|---|---|---|
| 2 × TaqMan Master Mix | 25 | μl × n |
| Forward primer (100 pmol/ml) | 0.1 | μl × n |
| Reverse primer (100 pmol/ml) | 0.1 | μl × n |
| Probe (100 pmol/ml) | 0.1 | μl × n |
| H₂O | 19.7 | μl × n |

TABLE 6
Components in the qPCRmix Master II

| | | |
|---|---|---|
| 2 × TaqMan Master Mix | 25 | μl × n |
| 10 × RNase P primer/probe mix | 2.5 | μl × n |
| H₂O | 17.5 | μl × n |

TABLE 7
Titration results of recombinant lentivirus

| Lentivirus | Tite |
|---|---|
| LV-BM38-07 | 9.3 E+10 |
| LV-BM38-06 | 1.2 E+11 |
| LV-BM38-05 | 4.5 E+11 |
| LV-38BM-07 | 8.9 E+10 |
| LV-38BM-06 | 6.7 E+10 |
| LV-38BM-05 | 2.4 E+10 |

TABLE 8
Titer of BCMA-Fc recombinant lentivirus-infected
Jurkar cells

| Lentivirus | Infection Rate | Tite (TU/mL) |
|---|---|---|
| LV-BM38-07 | 62.5% | 1.25 E+08 |
| LV-BM38-06 | 74.8% | 1.50 E+08 |
| LV-BM38-05 | 68.4% | 1.37 E+08 |
| LV-38BM-07 | 56.3% | 1.13 E+08 |
| LV-38BM-06 | 45.8% | 0.92 E+08 |
| LV-38BM-05 | 67.6% | 1.35 E+08 |

TABLE 9
Analysis of killing efficiency of BM38 CAR-T cells in vitro

| | CAR-T | | | | | | | | | |
|---|---|---|---|---|---|---|---|---|---|---|
| | effector- | | | | | | | | | |
| Cytotoxicity | to-target | BM38-07 | | | BM38-06 | | | BM38-05 | | |
| (%) | ratio | 0.5 | 2.5 | 12.5 | 0.5 | 2.5 | 12.5 | 0.5 | 2.5 | 12.5 |

| | | | | | | | | | |
|---|---|---|---|---|---|---|---|---|---|
| K562 | 12.5 | 15.1 | 22.4 | 14.8 | 15.8 | 24.2 | 15.6 | 18.4 | 22.8 |
| BCMA-K562 | 85.6 | 102.5 | 106.8 | 84.2 | 106.5 | 105.4 | 64.2 | 82.1 | 86.5 |

1. A chimeric antigen receptor, wherein: consisting of a signal peptide, two specific antigen-binding fragments, an extracellular spacer region, a transmembrane region, an intracellular co-stimulatory signaling domain, the first antigen that is recognized and bound by the specific antigen-binding fragments is a member selected from the group consisting of CD19, CD20, CD22, CD33, CD269, CD138, CD79a, CD79b, CD23, ROR1, CD30, B cell surface antibody light chain, CD44, CD123, Lewis Y, CD7 and CD46; the second antigen that is recognized and bound by the specific antigen-binding fragments is CD38, the two specific antigen-binding fragments is linked by a linker peptide.

2. The chimeric antigen receptor of claim 1, wherein: the chimeric antigen receptor is formed by a cell membrane localization signal peptide, a CD269 specific antigen-binding fragment, a linker peptide, a CD38 specific antigen-binding fragment, an extracellular spacer region, a transmembrane region, an intracellular signaling region and a co-stimulatory domain in a serially connected manner.

3. The chimeric antigen receptor of claim 2, wherein: the CD269 specific antigen-binding fragment comprises a framework region and a complementarity determining region CDR1-3, the amino acid sequence of the CD269 specific antigen-binding fragment includes any one of SEQ ID NO:1-SEQ ID NO:90 in the sequence list.

4. The chimeric antigen receptor of claim 3, wherein: the binding affinity constant Kd of the CD269 specific antigen-binding fragment to CD269 is <5.4×10⁻⁸M.

5. The chimeric antigen receptor of claim 2, wherein: the CD38 specific antigen-binding fragment comprises a framework region and a complementarity determining region CDR1-3, the, amino acid sequence of the CD38 specific antigen-binding fragment includes any one of SEQ ID NO:91-SEQ ID NO:180 in the sequence list.

6. The chimeric antigen receptor of claim 5, wherein: the binding affinity constant Kd of the CD38 specific antigen-binding fragment to CD38 is between 5.2×10⁻⁷M and 4.2×10⁻⁵M.

7. The chimeric antigen receptor of claim 2, wherein: the cell membrane localization signal peptide is a membrane localization signal peptide of CD4, CD8, G-CSFR or GM-CSFR.

8. The chimeric antigen receptor of claim 2, wherein: the linker peptide is (GGGGS)ₙ or (EAAAK)ₙ, wherein 1≤n<4.

9. The chimeric antigen receptor of claim 2, wherein: the extracellular spacer region is an extracellular domain of protein CD4, CD8, CD28, CD137, CD27, PD-1, OX40, TLR4, ICAM-1, ICOS(CD278), NKp80(KLRF1), NKp44, NKp30 or NKp46.

10. The chimeric antigen receptor of claim 2, wherein: the transmembrane region is a transmembrane domain of protein CD4, CD8, CD28, CD137, CD27, PD-1, IL2Rβ, IL2Rγ, IL7Rα, NKG2D, NKG2C, IgG4 or IgG1.

11. The chimeric antigen receptor of claim 2, wherein: the intracellular signaling region is an intracellular domain of CD2, CD3ζ, CD7, CD27, CD28, CD137, CD134, LCK, TNFR-1, TNFR-2, Fas, NKG-2D, DAP10, DAP12, B7-H3, TLR2 of TLR4IL7R or any combination thereof.

12. The chimeric antigen receptor of claim 1, wherein: the chimeric antigen receptor contains: (1) an amino acid sequence of any light chain variable region listed in SEQ ID NO:1-SEQ ID NO:180 in the sequence list; or (2) an amino acid sequence of any light chain variable region listed in SEQ ID NO:1-SEQ ID NO:180 in the sequence list, in which at least 1 but not more than 30 amino acid sequences are modified; or (3) an amino acid sequence having 90-99% identity to the amino acid sequence of any light chain variable region listed in SEQ ID NO:1-SEQ ID NO:180 in the sequence list.

13. The chimeric antigen receptor of claim 1, wherein: the chimeric antigen receptor contains: (1) an amino acid sequence of any heavy chain variable region listed in SEQ ID NO:1-SEQ ID NO:180 in the sequence list; or (2) an amino acid sequence of any heavy chain variable region listed in SEQ ID NO:1-SEQ ID NO:180 in the sequence list, in which at least 1 but not more than 30 amino acid sequences are modified; or (3) an amino acid sequence having 90-99% identity to the amino acid sequence of any heavy chain variable region listed in SEQ ID NO:1-SEQ ID NO:180 in the sequence list.

14. The chimeric antigen receptor of claim 2, wherein: the chimeric antigen receptor has an amino acid sequence corresponding to a construct in the Table 4.

15. A polypeptide, wherein: the polypeptide codes the chimeric antigen receptor of claim 1.

16. A gene, therein: the gene codes the chimeric antigen receptor of claim 1.

17. A genetically-engineered virus, wherein: the virus expresses the chimeric antigen receptor of claim 1 in a host cell.

18. A genetically-engineered effector cell, wherein: the effector cell expresses the polypeptide sequence of the chimeric antigen receptor of claim 1, the effector cell is a member selected from the group consisting of T lymphocytes, NK cells, hematopoietic stem cells, pluripotent stem cells, embryonic stem cells and induced pluripotent stem cells, or T lymphocytes and NK cells which is formed by inducing, culturing and differentiating the stem cells.

19. The genetically-engineered effector cell of claim 18, wherein the chimeric antigen receptor expresses on the cell membrane of the effector cell, and may specifically bind to the corresponding antigen.

20. Use of the chimeric antigen receptor of claim 1 in the preparation of antitumor drugs, anti-autoimmune disease drugs or anti-viral infectious disease drugs.


SEQUENCE LISTING (196 sequences)

SEQ ID NO: 1 - protein, 7 aa, Artificial Sequence
  Synthetic
GFTFSGY

SEQ ID NO: 2 - protein, 6 aa, Artificial Sequence
  Synthetic
NPDGSS

SEQ ID NO: 3 - protein, 7 aa, Artificial Sequence
  Synthetic
DYYGFDI

SEQ ID NO: 4 - protein, 11 aa, Artificial Sequence
  Synthetic
QGDSLRTYHAS

SEQ ID NO: 5 - protein, 7 aa, Artificial Sequence
  Synthetic
GKDNRPS

SEQ ID NO: 6 - protein, 11 aa, Artificial Sequence
  Synthetic
YSRDSSGNHFV

SEQ ID NO: 7 - protein, 116 aa, Artificial Sequence
  Synthetic
QVNLRESGGGLVQPGGSLRLSCAASGFTFSGYWMHWVRQAPGEGLVSVSRINPDGSSTIYADSVKGRFTISRDNAKNTLYLQMNSLKTEDTAVYYCSRDYYGFDIWGQGTMVTVSS

SEQ ID NO: 8 - protein, 107 aa, Artificial Sequence
  Synthetic
ELTQDPAVSVAVGQTVRLTCQGDSLRTYHASWYQQRPGQAPLLVFFGKDNRPSGIPDRFSGSSSGNTASLTITGAQAEDEADYYCYSRDSSGNHFVFGTGTKVTVLG

SEQ ID NO: 9 - protein, 238 aa, Artificial Sequence
  Synthetic
QVNLRESGGGLVQPGGSLRLSCAASGFTFSGYWMHWVRQAPGEGLVSVSRINPDGSSTIYADSVKGRFTISRDNAKNTLYLQMNSLKTEDTAVYYCSRDYYGFDIWGQGTMVTVSSGGGGSGGGGSGGGGSELTQDPAVSVAVGQTVRLTCQGDSLRTYHASWYQQRPGQAPLLVFFGKDNRPSGIPDRFSGSSSGNTASLTITGAQAEDEADYYCYSRDSSGNHFVFGTGTKVTVLG

SEQ ID NO: 10 - protein, 7 aa, Artificial Sequence
  Synthetic
GFTFSSY

SEQ ID NO: 11 - protein, 6 aa, Artificial Sequence
  Synthetic
SGSGGS

SEQ ID NO: 12 - protein, 15 aa, Artificial Sequence
  Synthetic
GFLRRDGYNTNAFDV

SEQ ID NO: 13 - protein, 11 aa, Artificial Sequence
  Synthetic
RASQSISHYLA

SEQ ID NO: 14 - protein, 7 aa, Artificial Sequence
  Synthetic
DTSKRAT

SEQ ID NO: 15 - protein, 8 aa, Artificial Sequence
  Synthetic
QQRSIWRT

SEQ ID NO: 16 - protein, 124 aa, Artificial Sequence
  Synthetic
QVNLRESGGGLVQPGGSLRLSCAASGFTFSSYAMSWVRQAPGKGLEWVSAISGSGGSTYYADSVKGRFTISRDNSKNTLYLQVNSLRVEDTAVYYCARGFLRRDGYNTNAFDVWGQGTMVTVSS

SEQ ID NO: 17 - protein, 107 aa, Artificial Sequence
  Synthetic
DVVMTQSPATLSVSPGERATLSCRASQSISHYLAWYQQKPGQAPRLLIYDTSKRATGIPARFSGSGSGTDFTLTISSLEPDDFATYYCQQRSIWRTFGQGTKVEIKR

SEQ ID NO: 18 - protein, 246 aa, Artificial Sequence
  Synthetic
QVNLRESGGGLVQPGGSLRLSCAASGFTFSSYAMSWVRQAPGKGLEWVSAISGSGGSTYYADSVKGRFTISRDNSKNTLYLQVNSLRVEDTAVYYCARGFLRRDGYNTNAFDVWGQGTMVTVSSGGGGSGGGGSGGGGSDVVMTQSPATLSVSPGERATLSCRASQSISHYLAWYQQKPGQAPRLLIYDTSKRATGIPARFSGSGSGTDFTLTISSLEPDDFATYYCQQRSIWRTFGQGTKVEIKR

SEQ ID NO: 19 - protein, 9 aa, Artificial Sequence
  Synthetic
GGSISSSGY

SEQ ID NO: 20 - protein, 5 aa, Artificial Sequence
  Synthetic
NHSGS

SEQ ID NO: 21 - protein, 9 aa, Artificial Sequence
  Synthetic
SAYWTERDY

SEQ ID NO: 22 - protein, 11 aa, Artificial Sequence
  Synthetic
RASQSLRSNLA

SEQ ID NO: 23 - protein, 7 aa, Artificial Sequence
  Synthetic
DASRRAT

SEQ ID NO: 24 - protein, 9 aa, Artificial Sequence
  Synthetic
QQSGGKPST

SEQ ID NO: 25 - protein, 119 aa, Artificial Sequence
  Synthetic
QVQLQESGAGLLKPSETLSLTCTVSGGSISSSGYYWSWIRQPPGKGLEWIGEINHSGSTNYNPSLKSRVTISVDTSKNQFSLKLSSVTAADTAVYYCARSAYWTERDYWGQGTLVTVSS

SEQ ID NO: 26 - protein, 120 aa, Artificial Sequence
  Synthetic
ITSYSIHYTKLSKIVLTQSPGTLSLSPGERATLYCRASQSLRSNLAWYQQKPGQAPRLLIYDASRRATGIPDRFSGSGSGTDFTLTISSLQPEDFATYYCQQSGGKPSTFGQGTKVEIKR

SEQ ID NO: 27 - protein, 254 aa, Artificial Sequence
  Synthetic
QVQLQESGAGLLKPSETLSLTCTVSGGSISSSGYYWSWIRQPPGKGLEWIGEINHSGSTNYNPSLKSRVTISVDTSKNQFSLKLSSVTAADTAVYYCARSAYWTERDYWGQGTLVTVSSGGGGSGGGGSGGGGSITSYSIHYTKLSKIVLTQSPGTLSLSPGERATLYCRASQSLRSNLAWYQQKPGQAPRLLIYDASRRATGIPDRFSGSGSGTDFTLTISSLQPEDFATYYCQQSGGKPSTFGQGTKVEIKR

SEQ ID NO: 28 - protein, 7 aa, Artificial Sequence
  Synthetic
GFTFSNF

SEQ ID NO: 29 - protein, 6 aa, Artificial Sequence
  Synthetic
TTGGGD

SEQ ID NO: 30 - protein, 12 aa, Artificial Sequence
  Synthetic
HGYYDGYHLFDY

SEQ ID NO: 31 - protein, 11 aa, Artificial Sequence
  Synthetic
RANQGISNNLN

SEQ ID NO: 32 - protein, 7 aa, Artificial Sequence
  Synthetic
YTSNLQS

SEQ ID NO: 33 - protein, 9 aa, Artificial Sequence
  Synthetic
QQFTSLPYT

SEQ ID NO: 34 - protein, 121 aa, Artificial Sequence
  Synthetic
EVQLVESGGGLVQPGGSLRLSCAASGFTFSNFDMAWVRQAPGKGLVWVSSITTGGGDTYYADSVKGRFTISRDNAKSTLYLQMDSLRSEDTAVYYCVRHGYYDGYHLFDYWGQGTLVTVSS

SEQ ID NO: 35 - protein, 107 aa, Artificial Sequence
  Synthetic
DIQMTQSPSSLSASVGDRVTITCRANQGISNNLNWYQQKPGKAPKPLIYYTSNLQSGVPSRFSGSGSGTDYTLTISSLQPEDFATYYCQQFTSLPYTFGQGTKLEIK

SEQ ID NO: 36 - protein, 243 aa, Artificial Sequence
  Synthetic
EVQLVESGGGLVQPGGSLRLSCAASGFTFSNFDMAWVRQAPGKGLVWVSSITTGGGDTYYADSVKGRFTISRDNAKSTLYLQMDSLRSEDTAVYYCVRHGYYDGYHLFDYWGQGTLVTVSSGGGGSGGGGSGGGGSDIQMTQSPSSLSASVGDRVTITCRANQGISNNLNWYQQKPGKAPKPLIYYTSNLQSGVPSRFSGSGSGTDYTLTISSLQPEDFATYYCQQFTSLPYTFGQGTKLEIK

SEQ ID NO: 37 - protein, 7 aa, Artificial Sequence
  Synthetic
GFNFNDY

SEQ ID NO: 38 - protein, 6 aa, Artificial Sequence
  Synthetic
WHDGSQ

SEQ ID NO: 39 - protein, 10 aa, Artificial Sequence
  Synthetic
PALLEVIFDD

SEQ ID NO: 40 - protein, 13 aa, Artificial Sequence
  Synthetic
RSSQSLLDSDDGN

SEQ ID NO: 41 - protein, 7 aa, Artificial Sequence
  Synthetic
TLSYRAS

SEQ ID NO: 42 - protein, 9 aa, Artificial Sequence
  Synthetic
MQGLQNPIT

SEQ ID NO: 43 - protein, 119 aa, Artificial Sequence
  Synthetic
QVQLVQSGGGVVQPGRSLRLSCTASGFNFNDYGMYWVRQAPGKGLEWVANIWHDGSQRHYAASVQGRFTTSRDNSRNTVYLQMNSLRAEDTALYYCVRPALLEVIFDDWGQGTLVTVSS

SEQ ID NO: 44 - protein, 114 aa, Artificial Sequence
  Synthetic
DIQMTQSPLSLPVTPGEPASISCRSSQSLLDSDDGNTYLDWYLQKPGQSPQLLIYTLSYRASGVPDRFSGSGSGTDFTLKISRVEAEDVGIYYCMQGLQNPITFGQGTRLEIKR

SEQ ID NO: 45 - protein, 248 aa, Artificial Sequence
  Synthetic
QVQLVQSGGGVVQPGRSLRLSCTASGFNFNDYGMYWVRQAPGKGLEWVANIWHDGSQRHYAASVQGRFTTSRDNSRNTVYLQMNSLRAEDTALYYCVRPALLEVIFDDWGQGTLVTVSSGGGGSGGGGSGGGGSDIQMTQSPLSLPVTPGEPASISCRSSQSLLDSDDGNTYLDWYLQKPGQSPQLLIYTLSYRASGVPDRFSGSGSGTDFTLKISRVEAEDVGIYYCMQGLQNPITFGQGTRLEIKR

SEQ ID NO: 46 - protein, 7 aa, Artificial Sequence
  Synthetic
GLTFSSY

SEQ ID NO: 47 - protein, 6 aa, Artificial Sequence
  Synthetic
SVTGGT

SEQ ID NO: 48 - protein, 9 aa, Artificial Sequence
  Synthetic
MKGQLVGSF

SEQ ID NO: 49 - protein, 11 aa, Artificial Sequence
  Synthetic
RASQSISTYLN

SEQ ID NO: 50 - protein, 7 aa, Artificial Sequence
  Synthetic
GASSLQR

SEQ ID NO: 51 - protein, 9 aa, Artificial Sequence
  Synthetic
QQYDNLPLT

SEQ ID NO: 52 - protein, 120 aa, Artificial Sequence
  Synthetic
QVQLQESGGGLVQPGGSLRLSCAASGLTFSSYAMGWVRQAPGKGLEWVSSISVTGGTTYHAASVRGRFTISRDNSKNTLYLQMNSLRADDTAIYYCVKMKGQLVGSFDYWGQGTLVTVSS

SEQ ID NO: 53 - protein, 108 aa, Artificial Sequence
  Synthetic
EIVLTQSPSSLSASVGDRVTITCRASQSISTYLNWYQQKPGKAPNLLIYGASSLQRGVPSRFSGSGSGTEFTLTISSLQPEDIATYYCQQYDNLPLTFGGGTKLEIKR

SEQ ID NO: 54 - protein, 243 aa, Artificial Sequence
  Synthetic
QVQLQESGGGLVQPGGSLRLSCAASGLTFSSYAMGWVRQAPGKGLEWVSSISVTGGTTYHAASVRGRFTISRDNSKNTLYLQMNSLRADDTAIYYCVKMKGQLVGSFDYWGQGTLVTVSSGGGGSGGGGSGGGGSEIVLTQSPSSLSASVGDRVTITCRASQSISTYLNWYQQKPGKAPNLLIYGASSLQRGVPSRFSGSGSGTEFTLTISSLQPEDIATYYCQQYDNLPLTFGGGTKLEIKR

SEQ ID NO: 55 - protein, 7 aa, Artificial Sequence
  Synthetic
GYSFSTY

SEQ ID NO: 56 - protein, 6 aa, Artificial Sequence
  Synthetic
SYDGSN

SEQ ID NO: 57 - protein, 8 aa, Artificial Sequence
  Synthetic
HKEINFDY

SEQ ID NO: 58 - protein, 11 aa, Artificial Sequence
  Synthetic
RASQSISSRLS

SEQ ID NO: 59 - protein, 7 aa, Artificial Sequence
  Synthetic
SASSLQT

SEQ ID NO: 60 - protein, 9 aa, Artificial Sequence
  Synthetic
QQSYTTPRT

SEQ ID NO: 61 - protein, 117 aa, Artificial Sequence
  Synthetic
QVQLQESGGGVVQPGRSLRLSCAASGYSFSTYAIHWVRQAPGKGLEWVAVISYDGSNKNYADSVKGRFTISRDNSKSTLYLDMNSLRAEDTAVYYCATHKEINFDYWGQGTLVTVSS

SEQ ID NO: 62 - protein, 108 aa, Artificial Sequence
  Synthetic
DVVMTQSPASLSASVGDRVTITCRASQSISSRLSWYQQKPGKAPKLLIYSASSLQTGVPSRFSGGGSRTDFTLTISNLQPEDFAPYYCQQSYTTPRTFGQGTKVDIKR

SEQ ID NO: 63 - protein, 240 aa, Artificial Sequence
  Synthetic
QVQLQESGGGVVQPGRSLRLSCAASGYSFSTYAIHWVRQAPGKGLEWVAVISYDGSNKNYADSVKGRFTISRDNSKSTLYLDMNSLRAEDTAVYYCATHKEINFDYWGQGTLVTVSSGGGGSGGGGSGGGGSDVVMTQSPASLSASVGDRVTITCRASQSISSRLSWYQQKPGKAPKLLIYSASSLQTGVPSRFSGGGSRTDFTLTISNLQPEDFAPYYCQQSYTTPRTFGQGTKVDIKR

SEQ ID NO: 64 - protein, 7 aa, Artificial Sequence
  Synthetic
GFTFSAY

SEQ ID NO: 65 - protein, 6 aa, Artificial Sequence
  Synthetic
SASGTS

SEQ ID NO: 66 - protein, 10 aa, Artificial Sequence
  Synthetic
GGAYRTALDS

SEQ ID NO: 67 - protein, 12 aa, Artificial Sequence
  Synthetic
RASQSVSSSYLA

SEQ ID NO: 68 - protein, 7 aa, Artificial Sequence
  Synthetic
AASNRAT

SEQ ID NO: 69 - protein, 8 aa, Artificial Sequence
  Synthetic
QQCSTPRT

SEQ ID NO: 70 - protein, 119 aa, Artificial Sequence
  Synthetic
QVNLRESGGGVVQPGRSLRLSCAASGFTFSAYGMSWVRQAPGKGLEWVAVTSASGTSTFHADSVKGRFTISRDNAKTTVFLQLNNLRDDDTAVYYCARGGAYRTALDSWGHGTLVTVSS

SEQ ID NO: 71 - protein, 108 aa, Artificial Sequence
  Synthetic
EIVLTQSPGTLSLSPGERATLSCRASQSVSSSYLAWYQQKPGQAPRLLIYAASNRATGIPDRFSGNGSGTDFTLTVSRLEPEDFAVYYCQQCSTPRTFGQGTKLEIKR

SEQ ID NO: 72 - protein, 242 aa, Artificial Sequence
  Synthetic
QVNLRESGGGVVQPGRSLRLSCAASGFTFSAYGMSWVRQAPGKGLEWVAVTSASGTSTFHADSVKGRFTISRDNAKTTVFLQLNNLRDDDTAVYYCARGGAYRTALDSWGHGTLVTVSSGGGGSGGGGSGGGGSEIVLTQSPGTLSLSPGERATLSCRASQSVSSSYLAWYQQKPGQAPRLLIYAASNRATGIPDRFSGNGSGTDFTLTVSRLEPEDFAVYYCQQCSTPRTFGQGTKLEIKR

SEQ ID NO: 73 - protein, 7 aa, Artificial Sequence
  Synthetic
GYTFNTY

SEQ ID NO: 74 - protein, 6 aa, Artificial Sequence
  Synthetic
DPNDSS

SEQ ID NO: 75 - protein, 12 aa, Artificial Sequence
  Synthetic
GPKWELHNYFDY

SEQ ID NO: 76 - protein, 11 aa, Artificial Sequence
  Synthetic
QGDTVRNHFPS

SEQ ID NO: 77 - protein, 7 aa, Artificial Sequence
  Synthetic
GKNNRPS

SEQ ID NO: 78 - protein, 12 aa, Artificial Sequence
  Synthetic
NSRDTRGNQMGL

SEQ ID NO: 79 - protein, 121 aa, Artificial Sequence
  Synthetic
GVQLVESGAEAKKPGESLRISCQISGYTFNTYWISWLRQMPGKGPEWMGRIDPNDSSTDYSPSFQGHITISTDNSIRTAYLQWSSLRTSDTALYYCAKGPKWELHNYFDYWGQGTLVTVSS

SEQ ID NO: 80 - protein, 108 aa, Artificial Sequence
  Synthetic
ELTQDPAVSVALGQTVRITCQGDTVRNHFPSWYQQKPGQAPKVVLYGKNNRPSGVPDRFSGSNSGNTASLIITGAQAEDEADYYCNSRDTRGNQMGLFGTGTKVTVLG

SEQ ID NO: 81 - protein, 246 aa, Artificial Sequence
  Synthetic
GVQLVESGAEAKKPGESLRISCQISGYTFNTYWISWLRQMPGKGPEWMGRIDPNDSSTDYSPSFQGHITISTDNSIRTAYLQWSSLRTSDTALYYCAKGPKWELHNYFDYWGQGTLVTVSSGGGGSGGGGSGGGGSSSELTQDPAVSVALGQTVRITCQGDTVRNHFPSWYQQKPGQAPKVVLYGKNNRPSGVPDRFSGSNSGNTASLIITGAQAEDEADYYCNSRDTRGNQMGLFGTGTKVTVLG

SEQ ID NO: 82 - protein, 7 aa, Artificial Sequence
  Synthetic
GFTFSAY

SEQ ID NO: 83 - protein, 5 aa, Artificial Sequence
  Synthetic
SASGT

SEQ ID NO: 84 - protein, 10 aa, Artificial Sequence
  Synthetic
GGAYRTALDS

SEQ ID NO: 85 - protein, 12 aa, Artificial Sequence
  Synthetic
RASQSVSSSYLA

SEQ ID NO: 86 - protein, 7 aa, Artificial Sequence
  Synthetic
AASNRAT

SEQ ID NO: 87 - protein, 8 aa, Artificial Sequence
  Synthetic
QQCSTPRT

SEQ ID NO: 88 - protein, 119 aa, Artificial Sequence; identical to SEQ ID NO: 70
  Synthetic

SEQ ID NO: 89 - protein, 108 aa, Artificial Sequence; identical to SEQ ID NO: 71
  Synthetic

SEQ ID NO: 90 - protein, 242 aa, Artificial Sequence; identical to SEQ ID NO: 72
  Synthetic

SEQ ID NO: 91 - protein, 7 aa, Artificial Sequence
  Synthetic
GGTFSSY

SEQ ID NO: 92 - protein, 6 aa, Artificial Sequence
  Synthetic
IPILGI

SEQ ID NO: 93 - protein, 8 aa, Artificial Sequence
  Synthetic
QYSSSFDP

SEQ ID NO: 94 - protein, 11 aa, Artificial Sequence
  Synthetic
RASRSINKWLA

SEQ ID NO: 95 - protein, 7 aa, Artificial Sequence
  Synthetic
SASTLES

SEQ ID NO: 96 - protein, 8 aa, Artificial Sequence
  Synthetic
QQYHDYPT

SEQ ID NO: 97 - protein, 117 aa, Artificial Sequence
  Synthetic
QVQLLQSAAEVKKPGSSVKVSCKASGGTFSSYTISWVRQAPGQGLEWMGRIIPILGIANYAQKFQGRVTITADKSTSTAYMELSSLRSEDTAVYYCARQYSSSFDPWGQGTLVTVSS

SEQ ID NO: 98 - protein, 107 aa, Artificial Sequence
  Synthetic
DIVMTQSPSTLSASVGDRVTITCRASRSINKWLAWYQHKPGKVPKLLIFSASTLESGVPSRFSGSGSGTEFTLTINSLQPDDFATFYCQQYHDYPTFGQGTKVEIKR

SEQ ID NO: 99 - protein, 239 aa, Artificial Sequence
  Synthetic
QVQLLQSAAEVKKPGSSVKVSCKASGGTFSSYTISWVRQAPGQGLEWMGRIIPILGIANYAQKFQGRVTITADKSTSTAYMELSSLRSEDTAVYYCARQYSSSFDPWGQGTLVTVSSGGGGSGGGGSGGGGSDIVMTQSPSTLSASVGDRVTITCRASRSINKWLAWYQHKPGKVPKLLIFSASTLESGVPSRFSGSGSGTEFTLTINSLQPDDFATFYCQQYHDYPTFGQGTKVEIKR

SEQ ID NO: 100 - protein, 7 aa, Artificial Sequence
  Synthetic
GYTFASY

SEQ ID NO: 101 - protein, 6 aa, Artificial Sequence
  Synthetic
TPSSGN

SEQ ID NO: 102 - protein, 15 aa, Artificial Sequence
  Synthetic
GPQSGYTYYYYGLDV

SEQ ID NO: 103 - protein, 11 aa, Artificial Sequence
  Synthetic
RASQSISTWLA

SEQ ID NO: 104 - protein, 7 aa, Artificial Sequence
  Synthetic
KASSLES

SEQ ID NO: 105 - protein, 10 aa, Artificial Sequence
  Synthetic
QQYNSYSPET

SEQ ID NO: 106 - protein, 124 aa, Artificial Sequence
  Synthetic
QVQLVQSGAEVKKPGASVKVSCTASGYTFASYDINWVRQATGQGLEWMGWMTPSSGNTGYAQKFQGRVTMTRNTSISTAYMEVSSLRSEDTAVYYCARGPQSGYTYYYYGLDVWGQGTMVTVSS

SEQ ID NO: 107 - protein, 109 aa, Artificial Sequence
  Synthetic
EIVLTQSPSTLSASVGDRVTITCRASQSISTWLAWYKQKPGKAPELLIYKASSLESGVPSRFSGSGSGTEFTLTISSLQPDDFATYYCQQYNSYSPETFGQGTKVDIKR

SEQ ID NO: 108 - protein, 248 aa, Artificial Sequence
  Synthetic
QVQLVQSGAEVKKPGASVKVSCTASGYTFASYDINWVRQATGQGLEWMGWMTPSSGNTGYAQKFQGRVTMTRNTSISTAYMEVSSLRSEDTAVYYCARGPQSGYTYYYYGLDVWGQGTMVTVSSGGGGSGGGGSGGGGSEIVLTQSPSTLSASVGDRVTITCRASQSISTWLAWYKQKPGKAPELLIYKASSLESGVPSRFSGSGSGTEFTLTISSLQPDDFATYYCQQYNSYSPETFGQGTKVDIKR

SEQ ID NO: 109 - protein, 5 aa, Artificial Sequence
  Synthetic
GYSFT

SEQ ID NO: 110 - protein, 5 aa, Artificial Sequence
  Synthetic
YPGDP

SEQ ID NO: 111 - protein, 13 aa, Artificial Sequence
  Synthetic
SVSTVVTPGGFDS

SEQ ID NO: 112 - protein, 13 aa, Artificial Sequence
  Synthetic
TRSSGSIASNYVH

SEQ ID NO: 113 - protein, 7 aa, Artificial Sequence
  Synthetic
EDNHRPS

SEQ ID NO: 114 - protein, 9 aa, Artificial Sequence
  Synthetic
QSYDSTTWF

SEQ ID NO: 115 - protein, 122 aa, Artificial Sequence
  Synthetic
QVQLQQSGAEVKKPGESLKISCKASGYSFTSYWIGWVRQMPGKGLEWMGMIYPGDPEIRYSPSFQGQVSISADKSVNTAYLQWSSLKASDTAMYYCMRSVSTVVTPGGFDSWGQGTLVTVSS

SEQ ID NO: 116 - protein, 111 aa, Artificial Sequence
  Synthetic
NFMLTQPHSVSESPGKTVTITCTRSSGSIASNYVHWCQQRPGSAPTTVIFEDNHRPSGVPDRFSGSIDRSSNSASLTISGLQAEDEADYYCQSYDSTTWFFGTGTQLTVLS

SEQ ID NO: 117 - protein, 248 aa, Artificial Sequence
  Synthetic
QVQLQQSGAEVKKPGESLKISCKASGYSFTSYWIGWVRQMPGKGLEWMGMIYPGDPEIRYSPSFQGQVSISADKSVNTAYLQWSSLKASDTAMYYCMRSVSTVVTPGGFDSWGQGTLVTVSSGGGGSGGGGSGGGGSNFMLTQPHSVSESPGKTVTITCTRSSGSIASNYVHWCQQRPGSAPTTVIFEDNHRPSGVPDRFSGSIDRSSNSASLTISGLQAEDEADYYCQSYDSTTWFFGTGTQLTVLS

SEQ ID NO: 118 - protein, 7 aa, Artificial Sequence
  Synthetic
GFTFSSY

SEQ ID NO: 119 - protein, 6 aa, Artificial Sequence
  Synthetic
STGGST

SEQ ID NO: 120 - protein, 11 aa, Artificial Sequence
  Synthetic
ISGFYFYGMDV

SEQ ID NO: 121 - protein, 11 aa, Artificial Sequence
  Synthetic
QGDSLRIYYPG

SEQ ID NO: 122 - protein, 7 aa, Artificial Sequence
  Synthetic
GKNMRPS

SEQ ID NO: 123 - protein, 11 aa, Artificial Sequence
  Synthetic
NSRDSSGKRVL

SEQ ID NO: 124 - protein, 120 aa, Artificial Sequence
  Synthetic
QVQLVQSGGDLVQPGGSLRLSCAAPGFTFSSYEMNWVRQAPGKGLEWVSYISTGGSTIYYADSVKGRFTISRDNARDSLYLEMNSLRAEDTAVYYCARISGFYFYGMDVWGQGTMVTVSS

SEQ ID NO: 125 - protein, 107 aa, Artificial Sequence
  Synthetic
ELTQDPAVSVALGQTVRITCQGDSLRIYYPGWYQQKPGQAPILVIYGKNMRPSGVPDRFSGSRSGNTASLTITGSRAEDEADYYCNSRDSSGKRVLFGGGTKLTVLG

SEQ ID NO: 126 - protein, 244 aa, Artificial Sequence
  Synthetic
QVQLVQSGGDLVQPGGSLRLSCAAPGFTFSSYEMNWVRQAPGKGLEWVSYISTGGSTIYYADSVKGRFTISRDNARDSLYLEMNSLRAEDTAVYYCARISGFYFYGMDVWGQGTMVTVSSGGGGSGGGGSGGGGSSSELTQDPAVSVALGQTVRITCQGDSLRIYYPGWYQQKPGQAPILVIYGKNMRPSGVPDRFSGSRSGNTASLTITGSRAEDEADYYCNSRDSSGKRVLFGGGTKLTVLG

SEQ ID NO: 127 - protein, 8 aa, Artificial Sequence
  Synthetic
GGSISGGD

SEQ ID NO: 128 - protein, 5 aa, Artificial Sequence
  Synthetic
YHTGG

SEQ ID NO: 129 - protein, 12 aa, Artificial Sequence
  Synthetic
APDDTSPGGLDY

SEQ ID NO: 130 - protein, 11 aa, Artificial Sequence
  Synthetic
RAPQDIRNSLA

SEQ ID NO: 131 - protein, 7 aa, Artificial Sequence
  Synthetic
AASSLQS

SEQ ID NO: 132 - protein, 9 aa, Artificial Sequence
  Synthetic
QQYGDSPLT

SEQ ID NO: 133 - protein, 121 aa, Artificial Sequence
  Synthetic
QVQLQESGPGLVKPSGTLSLTCAVSGGSISGGDWWSWVRQPPGKGLEWIGEIYHTGGTNYNPSLKRRVTISVDTSRNQFSLQLTSVTAADTAVYYCMTAPDDTSPGGLDYWGQGTLVTVSS

SEQ ID NO: 134 - protein, 108 aa, Artificial Sequence
  Synthetic
DIVMTQSPSSLSASVGDRVTITCRAPQDIRNSLAWYQQKPGKAPKLLISAASSLQSGVPSRFSGSGSGTDFTLTISRLEPEDFAVYYCQQYGDSPLTVGGGTKVEIKR

SEQ ID NO: 135 - protein, 244 aa, Artificial Sequence
  Synthetic
QVQLQESGPGLVKPSGTLSLTCAVSGGSISGGDWWSWVRQPPGKGLEWIGEIYHTGGTNYNPSLKRRVTISVDTSRNQFSLQLTSVTAADTAVYYCMTAPDDTSPGGLDYWGQGTLVTVSSGGGGSGGGGSGGGGSDIVMTQSPSSLSASVGDRVTITCRAPQDIRNSLAWYQQKPGKAPKLLISAASSLQSGVPSRFSGSGSGTDFTLTISRLEPEDFAVYYCQQYGDSPLTVGGGTKVEIKR

SEQ ID NO: 136 - protein, 5 aa, Artificial Sequence
  Synthetic
GYSFT

SEQ ID NO: 137 - protein, 6 aa, Artificial Sequence
  Synthetic
YPGDSD

SEQ ID NO: 138 - protein, 18 aa, Artificial Sequence
  Synthetic
RLSIRRQMNWGPGDAFDL

SEQ ID NO: 139 - protein, 11 aa, Artificial Sequence
  Synthetic
RASQSVSKFLN

SEQ ID NO: 140 - protein, 7 aa, Artificial Sequence
  Synthetic
DVSTLQT

SEQ ID NO: 141 - protein, 9 aa, Artificial Sequence
  Synthetic
QQSYSTPLT

SEQ ID NO: 142 - protein, 127 aa, Artificial Sequence
  Synthetic
QVQLQQSGAEVKKPGESLKISCKGSGYSFTSYWIGWVRQMPGKGLEWMGIIYPGDSDTRYSPSFQGQVTISADKSISTAYLQWSCLKASDTAMYYCARRLSIRRQMNWGPGDAFDLWGQGTMVTVSS

SEQ ID NO: 143 - protein, 108 aa, Artificial Sequence
  Synthetic
EIVLTQSPSSLSASVGDRVTITCRASQSVSKFLNWYQLKPGKAPKLLIYDVSTLQTGVPSRFTGSRSGTDFTLTINSLQPEDFATYFCQQSYSTPLTFGGGTKLEIKR

SEQ ID NO: 144 - protein, 250 aa, Artificial Sequence
  Synthetic
QVQLQQSGAEVKKPGESLKISCKGSGYSFTSYWIGWVRQMPGKGLEWMGIIYPGDSDTRYSPSFQGQVTISADKSISTAYLQWSCLKASDTAMYYCARRLSIRRQMNWGPGDAFDLWGQGTMVTVSSGGGGSGGGGSGGGGSEIVLTQSPSSLSASVGDRVTITCRASQSVSKFLNWYQLKPGKAPKLLIYDVSTLQTGVPSRFTGSRSGTDFTLTINSLQPEDFATYFCQQSYSTPLTFGGGTKLEIKR

SEQ ID NO: 145 - protein, 7 aa, Artificial Sequence
  Synthetic
GGSISGY

SEQ ID NO: 146 - protein, 5 aa, Artificial Sequence
  Synthetic
HDTGG

SEQ ID NO: 147 - protein, 9 aa, Artificial Sequence
  Synthetic
GIYGAYFDS

SEQ ID NO: 148 - protein, 11 aa, Artificial Sequence
  Synthetic
RTSQSINRYLA

SEQ ID NO: 149 - protein, 7 aa, Artificial Sequence
  Synthetic
RASSRAT

SEQ ID NO: 150 - protein, 8 aa, Artificial Sequence
  Synthetic
QQYSDWPT

SEQ ID NO: 151 - protein, 117 aa, Artificial Sequence
  Synthetic
QVQLQESGPGLVKPSETLSLTCTLSGGSISGYYWSWIRQPPGKGLEWIAYIHDTGGIEYYPSLKTRLTISSDASRNQFSLKLSSVTAADTAVYYCARGIYGAYFDSWGQGTLVTVSS

SEQ ID NO: 152 - protein, 239 aa, Artificial Sequence
  Synthetic
QVQLQESGPGLVKPSETLSLTCTLSGGSISGYYWSWIRQPPGKGLEWIAYIHDTGGIEYYPSLKTRLTISSDASRNQFSLKLSSVTAADTAVYYCARGIYGAYFDSWGQGTLVTVSSGGGGSGGGGSGGGGSEIVLTQSPATLSLSPGERATLSCRTSQSINRYLAWYQQKLGQAPRLLIYRASSRATGIPDRFSGSGSGTDFTLTISRLEPEDFGVYYCQQYSDWPTFGGGTKVEIKR

SEQ ID NO: 153 - protein, 7 aa, Artificial Sequence
  Synthetic
GGTFSSY

SEQ ID NO: 154 - protein, 6 aa, Artificial Sequence
  Synthetic
IIRFLG

SEQ ID NO: 155 - protein, 12 aa, Artificial Sequence
  Synthetic
EPGERDPDAVDI

SEQ ID NO: 156 - protein, 7 aa, Artificial Sequence
  Synthetic
GGTFSSY

SEQ ID NO: 157 - protein, 6 aa, Artificial Sequence
  Synthetic
IIRFLG

SEQ ID NO: 158 - protein, 12 aa, Artificial Sequence
  Synthetic
EPGERDPDAVDI

SEQ ID NO: 159 - protein, 107 aa, Artificial Sequence
  Synthetic
EIVLTQSPATLSLSPGERATLSCRTSQSINRYLAWYQQKLGQAPRLLIYRASSRATGIPDRFSGSGSGTDFTLTISRLEPEDFGVYYCQQYSDWPTFGGGTKVEIKR

SEQ ID NO: 160 - protein, 124 aa, Artificial Sequence
  Synthetic
QVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGEPGERDPDAVDIWGQGTMVTVSS

SEQ ID NO: 161 - protein, 107 aa, Artificial Sequence
  Synthetic
DIQMTQSPSSLSASVGDRVTITCRASQGISNYLAWFQQKPGKAPKSLIYAASSLQSGVPSRFSGSGSGTDFTLTISSLQPEDFATYYCQQYNSYPYTFGQGTKLEIK

SEQ ID NO: 162 - protein, 246 aa, Artificial Sequence
  Synthetic
QVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGEPGERDPDAVDIWGQGTMVTVSSGGGGSGGGGSGGGGSDIQMTQSPSSLSASVGDRVTITCRASQGISNYLAWFQQKPGKAPKSLIYAASSLQSGVPSRFSGSGSGTDFTLTISSLQPEDFATYYCQQYNSYPYTFGQGTKLEIK

SEQ ID NO: 163 - protein, 7 aa, Artificial Sequence
  Synthetic
GGSISSL

SEQ ID NO: 164 - protein, 5 aa, Artificial Sequence
  Synthetic
RYSGK

SEQ ID NO: 165 - protein, 11 aa, Artificial Sequence
  Synthetic
DSGGGYNWFDP

SEQ ID NO: 166 - protein, 11 aa, Artificial Sequence
  Synthetic
RASQSISTYLN

SEQ ID NO: 167 - protein, 7 aa, Artificial Sequence
  Synthetic
AASSLQG

SEQ ID NO: 168 - protein, 9 aa, Artificial Sequence
  Synthetic
QQSYNAPRT

SEQ ID NO: 169 - protein, 119 aa, Artificial Sequence
  Synthetic
QVQLQQSGPGLVKPSETLSLTCTVSGGSISSLQWNWIRQPLGKGLEWIGFVRYSGKNSYNPSLGSRVTMSLDMSKNQFSLNLSSLTAADTAVYYCARDSGGGYNWFDPWGQGTLVTVSS

SEQ ID NO: 170 - protein, 108 aa, Artificial Sequence
  Synthetic
EIVLTQSPDFQSVTPKEKVTITCRASQSISTYLNWYQQKPGKAPKILISAASSLQGGVPSRFSGSGSGTDFTLIISNLQPEDFASYYCQQSYNAPRTFGQGTKVEIKR

SEQ ID NO: 171 - protein, 242 aa, Artificial Sequence
  Synthetic
QVQLQQSGPGLVKPSETLSLTCTVSGGSISSLQWNWIRQPLGKGLEWIGFVRYSGKNSYNPSLGSRVTMSLDMSKNQFSLNLSSLTAADTAVYYCARDSGGGYNWFDPWGQGTLVTVSSGGGGSGGGGSGGGGSEIVLTQSPDFQSVTPKEKVTITCRASQSISTYLNWYQQKPGKAPKILISAASSLQGGVPSRFSGSGSGTDFTLIISNLQPEDFASYYCQQSYNAPRTFGQGTKVEIKR

SEQ ID NO: 172 - protein, 7 aa, Artificial Sequence
  Synthetic
GFTFSNY

SEQ ID NO: 173 - protein, 6 aa, Artificial Sequence
  Synthetic
SGSGIN

SEQ ID NO: 174 - protein, 6 aa, Artificial Sequence
  Synthetic
HGGGSF

SEQ ID NO: 175 - protein, 11 aa, Artificial Sequence
  Synthetic
RASQNMNSYLN

SEQ ID NO: 176 - protein, 7 aa, Artificial Sequence
  Synthetic
AASSLQS

SEQ ID NO: 177 - protein, 9 aa, Artificial Sequence
  Synthetic
QQANSFPYT

SEQ ID NO: 178 - protein, 115 aa, Artificial Sequence
  Synthetic
QVQLQQSGGGLVQPGGSLRLSCAASGFTFSNYAIIWVRQAPGKGLEWVSVISGSGINTYYADSVKGRFTISRDNSKNTVYLQLNSLRAEDTAIYYCVGHGGGSFWGQGTLVTVSS

SEQ ID NO: 179 - protein, 108 aa, Artificial Sequence
  Synthetic
EIVLTQSPSSLSASVGDRVTITCRASQNMNSYLNWYQQKPGKSPKVLIYAASSLQSGVPSRFSGSGSGTDFTLTISSLQPEDFATYYCQQANSFPYTFGPGTKVEIKR

SEQ ID NO: 180 - protein, 238 aa, Artificial Sequence
  Synthetic
QVQLQQSGGGLVQPGGSLRLSCAASGFTFSNYAIIWVRQAPGKGLEWVSVISGSGINTYYADSVKGRFTISRDNSKNTVYLQLNSLRAEDTAIYYCVGHGGGSFWGQGTLVTVSSGGGGSGGGGSGGGGSEIVLTQSPSSLSASVGDRVTITCRASQNMNSYLNWYQQKPGKSPKVLIYAASSLQSGVPSRFSGSGSGTDFTLTISSLQPEDFATYYCQQANSFPYTFGPGTKVEIKR

SEQ ID NO: 181 - protein, 22 aa, Artificial Sequence
  Synthetic
MLLLVTSLLLCELPHPAFLLIP

SEQ ID NO: 182 - protein, 45 aa, Artificial Sequence
  Synthetic
TTTPAPRPPTPAPTIASQPLSLRPEACRPAAGGAVHTRGLDFACD

SEQ ID NO: 183 - protein, 24 aa, Artificial Sequence
  Synthetic
IYIWAPLAGTCGVLLLSLVITLYC

SEQ ID NO: 184 - protein, 42 aa, Artificial Sequence
  Synthetic
KRGRKKLLYIFKQPFMRPVQTTQEEDGCSCRFPEEEEGGCEL

SEQ ID NO: 185 - protein, 68 aa, Artificial Sequence
  Synthetic
FWVLVVVGGVLACYSLLVTVAFIIFWVRSKRSRGGHSDYMNMTPRRPGPTRKHYQPYAPPRDFAAYRS

SEQ ID NO: 186 - protein, 112 aa, Artificial Sequence
  Synthetic
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 187 - DNA, 21 bp, Artificial Sequence
  Synthetic
atgttgcagatggctgggcag

SEQ ID NO: 188 - DNA, 21 bp, Artificial Sequence
  Synthetic
tacctagcagaaattgatttc

SEQ ID NO: 189 - DNA, 18 bp, Artificial Sequence
  Synthetic
tgtgatcatgttgcagat

SEQ ID NO: 190 - DNA, 18 bp, Artificial Sequence
  Synthetic
tacctagcagaaattgat

SEQ ID NO: 191 - DNA, 25 bp, Artificial Sequence
  Synthetic
tatcgatgctccggtgcccgtcagt

SEQ ID NO: 192 - DNA, 22 bp, Artificial Sequence
  Synthetic
tcacgacacctgaaatggaaga

SEQ ID NO: 193 - DNA, 17 bp, Artificial Sequence
  Synthetic
tccgggactttcgcttt

SEQ ID NO: 194 - DNA, 19 bp, Artificial Sequence
  Synthetic
cagaatccaggtggcaaca

SEQ ID NO: 195 - DNA, 21 bp, Artificial Sequence
  Synthetic
catgtacgttgctatccaggc

SEQ ID NO: 196 - DNA, 20 bp, Artificial Sequence
  Synthetic
tccttaatgtcacgcacgat